SEQUENCE LISTING (25 sequences)

SEQ ID NO: 1 - protein, 355 aa, Artificial Sequence
  Description of Artificial Sequence Synthetic polypeptide
MAKLTALTLLGLGLALFDGQKSSFQTRFNVHREVTPVELPNCNLVKGVDNGSEDLEILPNGLAFISSGLKYPGIMSFDPDKSGKILLMDLNEEDPVVLELGITGNTLDISSFNPHGISTFTDEDNTVYLLVVNHPDSSSTVEVFKFQEEEKSLLHLKTIRHKLLPSVNDIVAVGPEHFYATNDHYFADPYLKSWEMHLGLAWSFVTYYSPNDVRVVAEGFDFANGINISPDGKYVYIAELLAHKIHVYEKHANWTLTPLKSLDFDTLVDNISVDPVTGDLWVGCHPNGMRIFYYDPKNPPGSEVLRIQDILSEEPKVTVVYAENGTVLQGSTVAAVYKGKLLIGTVFHKALYCEL

SEQ ID NO: 2 - protein, 11 aa, Artificial Sequence
  Description of Artificial Sequence Synthetic peptide
SSGLKYPGIMS

SEQ ID NO: 3 - protein, 11 aa, Artificial Sequence
  Description of Artificial Sequence Synthetic peptide
KYPGIMSFDPD

SEQ ID NO: 4 - protein, 11 aa, Artificial Sequence
  Description of Artificial Sequence Synthetic peptide
GKILLMDLNEE

SEQ ID NO: 5 - protein, 11 aa, Artificial Sequence
  Description of Artificial Sequence Synthetic peptide
EDNTVYLLVVN

SEQ ID NO: 6 - protein, 11 aa, Artificial Sequence
  Description of Artificial Sequence Synthetic peptide
GPEHFYATNDH

SEQ ID NO: 7 - protein, 11 aa, Artificial Sequence
  Description of Artificial Sequence Synthetic peptide
ATNDHYFADPY

SEQ ID NO: 8 - protein, 11 aa, Artificial Sequence
  Description of Artificial Sequence Synthetic peptide
YFADPYLKSWE

SEQ ID NO: 9 - protein, 11 aa, Artificial Sequence
  Description of Artificial Sequence Synthetic peptide
PYLKSWEMHLG

SEQ ID NO: 10 - protein, 11 aa, Artificial Sequence
  Description of Artificial Sequence Synthetic peptide
LKSWEMHLGLA

SEQ ID NO: 11 - protein, 11 aa, Artificial Sequence
  Description of Artificial Sequence Synthetic peptide
LLGLAWSFVTY

SEQ ID NO: 12 - protein, 12 aa, Artificial Sequence
  Description of Artificial Sequence Synthetic peptide
WSFVTYYSPNDV

SEQ ID NO: 13 - protein, 12 aa, Artificial Sequence
  Description of Artificial Sequence Synthetic peptide
SPDGKYVYIAEL

SEQ ID NO: 14 - protein, 11 aa, Artificial Sequence
  Description of Artificial Sequence Synthetic peptide
DGKYVYIAELL

SEQ ID NO: 15 - protein, 11 aa, Artificial Sequence
  Description of Artificial Sequence Synthetic peptide
HKIHVYEKHAN

SEQ ID NO: 16 - protein, 10 aa, Artificial Sequence
  Description of Artificial Sequence Synthetic peptide
KHANWTLTPL

SEQ ID NO: 17 - protein, 11 aa, Artificial Sequence
  Description of Artificial Sequence Synthetic peptide
VTGDLWVGCHP

SEQ ID NO: 18 - protein, 11 aa, Artificial Sequence
  Description of Artificial Sequence Synthetic peptide
CHPNGMRIFYY

SEQ ID NO: 19 - protein, 12 aa, Artificial Sequence
  Description of Artificial Sequence Synthetic peptide
GMRIFYYDPKNP

SEQ ID NO: 20 - protein, 11 aa, Artificial Sequence
  Description of Artificial Sequence Synthetic peptide
KVTVVYAENGT

SEQ ID NO: 21 - protein, 11 aa, Artificial Sequence
  Description of Artificial Sequence Synthetic peptide
TVAAVYKGKLL

SEQ ID NO: 22 - protein, 9 aa, Artificial Sequence
  Description of Artificial Sequence Synthetic peptide
FHKALYCEL

SEQ ID NO: 23 - protein, 11 aa, Artificial Sequence
  Description of Artificial Sequence Synthetic peptide
YPGIMSFDPDK

SEQ ID NO: 24 - protein, 14 aa, Artificial Sequence
  Description of Artificial Sequence Synthetic peptide
YPGIMSFDPDKSGK

SEQ ID NO: 25 - protein, 10 aa, Artificial Sequence
  Description of Artificial Sequence Synthetic peptide
HANWTLTPLK

(19) United States
(12) Patent Application Publication
(10) Pub. No.: US 2011/0201947 A1
(43) Pub. Date: Aug. 18, 2011

(54) OXIDIZED PARAOXONASE 1 AND PARAOXONASE 1/HDL PARTICLE NUMBER RATIO AS RISK MARKERS FOR CARDIOVASCULAR DISEASE

(75) Inventors: Stanley L. Hazen, Pepper Pike, OH (US); Stephen James Nicholls, Cleveland Heights, OH (US); Zhiping Wu, Cleveland, OH (US)

(73) Assignee: THE CLEVELAND CLINIC FOUNDATION, Cleveland, OH (US)

(21) Appl. No.: 12/922,473

(22) PCT Filed: Mar. 16, 2009
(86) PCT No.: PCT/US09/37326

§ 371 Date: Mar. 7, 2011

Related U.S. Application Data
(60) Provisional application No. 61/036,562, filed on Mar. 14, 2008.
(60) Provisional application No. 61/036,566, filed on Mar. 14, 2008.

(51) Int. Cl. A61B 5/0205; C12Q 1/44; C07K 16/40; G01N 33/573

(52) U.S. Cl. 600/485; 435/19; 530/387.1; 435/7.1

(57) ABSTRACT

The present invention provides methods and markers for characterizing a subject's, particularly a human subject risk of having cardiovascular disease. The present invention also provides methods of characterizing a subject's risk of developing cardiovascular disease. In another embodiment, the present invention provides methods for characterizing a subject's risk of experiencing a complication of cardiovascular disease or major adverse cardiac event within 1, 3, or 10 years. In another embodiment, the present invention provides a method for determining whether a subject presenting with chest pain is at risk near term of experiencing a heart attack or other major adverse cardiac event. The present methods are especially useful for identifying those subjects who are in need of highly aggressive CVD therapies as well as those subjects who require no therapies targeted at inhibiting or preventing CVD or complications of CVD.

### CROSS-REFERENCE TO RELATED APPLICATIONS

This application claims priority to and the benefit of U.S. Provisional Application No. 61/036,562, filed Mar. 14, 2008 and U.S. Provisional Application No. 61/036,566, filed on Mar. 14, 2008, both of which are incorporated by reference herein in their entirety.

### STATEMENT REGARDING FEDERALLY SPONSORED RESEARCH

The work described in this application was supported, at least in part, by NIH Grant No. P01-HL-076491. The United States government may have certain rights in this invention.

### FIELD OF INVENTION

The present invention relates to the field of cardiovascular disease. More specifically, it relates to markers and methods for determining whether a subject, particularly a human subject, is at risk of developing cardiovascular disease, having cardiovascular disease, or experiencing a complication of cardiovascular disease, e.g., an adverse cardiac event. The present application also relates to the use of such markers and methods for monitoring the status of cardiovascular disease in a subject or the effects of therapeutic agents on subjects with cardiovascular disease.

### BACKGROUND

Cardiovascular disease (CVD) is the general term for heart and blood vessel diseases, including atherosclerosis, coronary heart disease, cerebrovascular disease, aortoiliac disease, and peripheral vascular disease. Subjects with CVD may develop a number of complications or experience a major adverse cardiac event (MACE), including, but not limited to, myocardial infarction, stroke, angina pectoris, transient ischemic attacks, congestive heart failure, aortic aneurysm, and death. CVD accounts for one in every two deaths in the United States and is the number one killer disease. Thus, prevention of cardiovascular disease is an area of major public health importance.

A low-fat diet and exercise are recommended to prevent CVD. In addition, a number of therapeutic agents may be prescribed by medical professionals to those individuals who are known to be at risk for developing or having CVD. These include lipid-lowering agents that reduce blood levels of cholesterol and triglycerides, agents that normalize blood pressure, agents, such as aspirin or platelet ADP receptor antagonist (e.g., clopidogrel and ticlopidine), that prevent activation of platelets and decrease vascular inflammation, and pleiotropic agents such as peroxisome proliferator activated receptor (PPAR) agonists, with broad-ranging metabolic effects that reduce inflammation, promote insulin sensitization, improve vascular function, and correct lipid abnormalities. More aggressive therapy, such as administration of multiple medications or surgical intervention may be used in those individuals who are at high risk. Since CVD therapies may have adverse side effects, it is desirable to have methods for identifying those individuals who are at risk, particularly those individuals who are at high risk, of developing or having CVD.

Currently, several risk factors are used by medical professionals to assess an individual's risk of developing or having CVD and to identify individuals at high risk. Major risk factors for cardiovascular disease include age, hypertension, family history of premature CVD, smoking, high total cholesterol, low HDL cholesterol, obesity and diabetes. The major risk factors for CVD are additive, and are typically used together by physicians in a risk prediction algorithm to target those individuals who are most likely to benefit from treatment for CVD. These algorithms achieve a high sensitivity and specificity for predicting risk of CVD within 10 years. However, the ability of the present algorithms to predict a higher probability of developing CVD is limited. Among those individuals with none of the current risk factors, the 10-year risk for developing CVD is still about 2%. In addition, a large number of CVD complications occur in individuals with apparently low to moderate risk profiles, as determined using currently known risk factors. Thus, there is a need to expand the present cardiovascular risk algorithm to identify a larger spectrum of individuals at risk for or affected with CVD.

The mechanism of atherosclerosis is not well understood. Over the past decade a wealth of clinical, pathological, biochemical and genetic data support the notion that atherosclerosis is a chronic inflammatory disorder. Acute phase reactants (e.g., C-reactive protein, complement proteins), sensitive but non-specific markers of inflammation, are enriched in fatty streaks and later stages of atherosclerotic lesions. In a recent prospective clinical trial, base-line plasma levels of C-reactive protein independently predicted risk of first-time myocardial infarction and stroke in apparently healthy individuals. U.S. Pat. No. 6,040,147 describes methods which use C-reactive protein, cytokines, and cellular adhesion molecules to characterize an individual's risk of developing a cardiovascular disorder. Although useful, these markers may be found in the blood of individuals with inflammation due to causes other than CVD, and thus, these markers may not be specific enough. Moreover, modulation of their levels has not been shown to predict a decrease in the morbidity or mortality of CVD.

### BRIEF SUMMARY OF THE INVENTION

The present invention provides methods and markers for characterizing a subject's, particularly a human subject's, risk of having cardiovascular disease. The present invention also provides methods of characterizing a subject's risk of developing cardiovascular disease. In another embodiment, the present invention provides methods for characterizing a subject's risk of experiencing a complication of cardiovascular disease or major adverse cardiac event within 1, 3, and 10 years. In another embodiment, the present invention provides a method for determining whether a subject presenting with chest pain is at risk near term of experiencing a heart attack or other major adverse cardiac event. The present methods are especially useful for identifying those subjects who are in need of highly aggressive CVD therapies, as well as those subjects who require no therapies targeted at inhibiting or preventing CVD or complications of CVD.

### Oxidized Paraoxonase 1 as a Risk Marker for Cardiovascular Disease

In one embodiment, the present methods comprise determining the levels of one or more oxidized biomolecules (referred to hereinafter collectively as “oxidized paraoxonase 1 (PON1)-related biomolecules”) in a bodily sample obtained from the subject. In one embodiment, the oxidized PON1-related biomolecule is an oxidized PON1 protein. In another embodiment, the oxidized PON1-related biomolecule is an oxidized PON1 peptide fragment. Levels of one or more of the oxidized PON1-related biomolecules in a biological sample from the subject may be compared to a control value that is derived from measurements of the one or more oxidized PON1-related biomolecules in comparable biological samples obtained from a population of control subjects. Levels of the one or more oxidized PON1-related biomolecules in a biological sample obtained from the subject, alternatively, may be compared to levels of an internal standard in the biological sample obtained from the subject. Examples of such internal standards include, but are not limited to, levels of total PON1 and/or total PON1 activity. Furthermore, levels of one or more oxidized PON1-related biomolecules in a biological sample obtained from the subject may be compared as a ratio of specific oxidation products to unoxidized precursor, for example the ratio of the level of oxidized PON1 to total PON1, and/or the ratio of oxidized PON1 activity to total PON1 activity.

In one embodiment, the comparison characterizes the subject's present risk of having CVD, as determined using standard protocols for diagnosing CVD. Moreover, the extent of the difference between a subject's oxidized PON1-related biomolecule levels and the control value is also useful for characterizing the extent of the risk and thereby determining which subjects would most greatly benefit from certain therapies. In another embodiment, the comparison characterizes the subject's risk of developing CVD in the future. In another embodiment, the comparison can be used to characterize the subject's risk of experiencing a complication of CVD or a major adverse cardiac event, such as myocardial infarction, reinfarction, need for revascularization, stroke, and/or death, within one, three, or 10 years after the sample is taken. The present methods can also be used to determine if a subject presenting with chest pain is at risk of experiencing a major adverse cardiac event, such as a myocardial infarction, reinfarction, need for revascularization, stroke and/or death, near term, e.g., within the following day, 3 months, or 6 months after the subject presents with chest pain.

Also provided herein are methods for monitoring over time the status of CVD in a subject. In one embodiment, the method comprises determining the levels of one or more of the oxidized PON1-related biomolecules in a biological sample taken from the subject at an initial time and in a corresponding biological sample taken from the subject at a subsequent time. An increase in levels of the one or more oxidized PON1-related biomolecules in a biological sample taken at the subsequent time as compared to the initial time indicates that a subject's risk of having CVD has increased. A decrease in levels of the one or more oxidized PON1-related molecules indicates that the subject's risk of having CVD has decreased. For those subjects who have already experienced an acute adverse cardiovascular event such as a myocardial infarction or ischemic stroke, such methods are also useful for assessing the subject's risk of experiencing a subsequent acute adverse cardiovascular event. In such subjects, an increase in levels of the one more oxidized PON1-related biomolecules indicates that the subject is at increased risk of experiencing a subsequent adverse cardiovascular event. A decrease in levels of the one or more oxidized PON1-related biomolecules in the subject over time indicates that the subject's risk of experiencing a subsequent adverse cardiovascular event has decreased.

In another embodiment, the present invention provides a method for characterizing a subject's response to therapy directed at stabilizing or regressing CVD. The method comprises determining levels of one or more oxidized PON1-related biomolecules in a biological sample taken from the subject prior to therapy and determining the level of the one or more of the oxidized PON1-related biomolecules in a corresponding biological sample taken from the subject during or following therapy. A decrease in levels of the one or more oxidized PON1-related biomolecules in the sample taken after or during therapy as compared to levels of the one or more oxidized PON1-related biomolecules in the sample taken before therapy is indicative of a positive effect of the therapy on cardiovascular disease in the treated subject.

In another embodiment, the present invention provides antibodies that are immunospecific for one or more of the oxidized PON1-related biomolecules used in the present methods. Such antibodies are useful for determining or measuring the levels of the oxidized PON 1-related biomolecules in biological samples obtained from the subject.

In another embodiment, the present invention relates to kits that comprise reagents for assessing levels of oxidized PON1 and/or oxidized PON1 peptide fragments in biological samples obtained from a test subject. The present kits also comprise printed materials such as instructions for practicing the present methods, or information useful for assessing a test subject's risk of CVD. Examples of such information include, but are not limited cut-off values, sensitivities at particular cut-off values, as well as other printed material for characterizing risk based upon the outcome of the assay. In some embodiments, such kits may also comprise control reagents, e.g., oxidized PON1.

### Paraoxonase 1/HDL Particle Number Ratio as a Risk Marker for Cardiovascular Disease

Human paraoxonase 1 (PON1) is a 43 kDa glycoprotein with a broad specificity class A esterase activity (La Du, B. N. et al., Chem Biol Interact. 1993 June; 87(1-3):25-34), capable of hydrolyzing a broad spectrum of organophosphate substrates and a number of aromatic carboxylic acid esters (Gan, K. N. et al., Drug Metab Dispos. 1991 January-February; 19(1):100-6). Recent studies suggest that this enzyme's arylesterase activity can hydrolyze bioactive oxidized phospholipids (Watson, A. D. et al., J Clin Invest. 1995 December; 96(6):2882-91) and lactones (Khersonsky, O. & Tawfik, D. S., Biochemistry. 2005 Apr. 26; 44(16):6371-82).

In one embodiment, the present methods comprise determining the ratio of paraoxonase 1 (PON1) activity to high density lipid (HDL) particle number in a biological sample, e.g., a bodily fluid obtained from the subject. Methods for measuring PON1 paraoxonase and arylesterase activity are described in Eckerson, H. W. et al., Am J Hum Genet. 1983 November; 35(6):1126-38 and Bhattacharyya, T. et al., JAMA 2008 Mar. 19; 299(11):1265-76. Methods for measuring PON1 lipolactonase activity are described in Gaidukov, L. & Tawfik, D. S., J Lipid Res. 2007 July; 48(7):1637-46. Thus, in certain embodiments, the present risk marker is a ratio of PON1 activity/HDL particle number (as determined by NMR), PON1 activity/apolipoprotein A-1 (apoA1) (a surrogate of HDL particle number), PON1 activity/apolipoprotein A-2 (apoA2) (another surrogate of HDL particle number), or PON1 activity/(apoA1+apoA2) that provides the greatest prognostic utility. In another embodiment, the present methods comprise determining the ratio of PON1 mass to HDL particle number, apoA1, apoA2, or (apoA1+apoA2) in a biological sample, for example, blood, serum, or plasma, from the subject.

### BRIEF DESCRIPTION OF THE DRAWINGS

FIG. 1 shows mass spectrometry data of human PON1 demonstrating site specific nitration and chlorination of Tyr71.

FIG. 2 shows that PON1 Tyr71 interacts with nascent HDL lipid (cholesterol).

FIG. 3 shows that PON1 and MPO reciprocally modulate each other's activity.

FIG. 4 shows a paraoxonase assay that indicates Tyr71 of PON1 is important for PON1 activity.

FIG. 5 shows an arylesterase assay that indicates Tyr71 of PON1 is important for PON1 activity.

FIG. 6 shows detection of oxidized Trp254 (hydroxytryptophan) in paraoxonase 1 (PON1).

FIG. 7 shows the percentage of patients who experienced a subsequent major adverse cardiovascular event during the next 3 years stratified according to baseline quartiles of PON1/apoA1.

FIG. 8 shows the percentage of patients who experienced a subsequent major adverse cardiovascular event during the next 3 years stratified according to baseline quartiles of PON1/HDL-C.

FIG. 9 shows mass spectrometry data of human PON1 demonstrating site specific chlorination of Tyr71 and oxidation of Met75.

FIG. 10 shows mass spectrometry data of human PON1 demonstrating site specific nitration of Tyr71 and oxidation of Met75.

FIG. 11 shows mass spectrometry data of human PON1 demonstrating site specific oxidation of Trp254.

FIG. 12 shows mass spectrometry data of human PON1 demonstrating site specific oxidation of Met75.

### DETAILED DESCRIPTION OF THE INVENTION

The present invention will now be described by reference to more detailed embodiments. This invention may, however, be embodied in different forms and should not be construed as limited to the embodiments set forth herein. Rather these embodiments are provided so that this disclosure will be thorough and complete, and will convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

All publications, patent applications, patents, and other references mentioned herein are incorporated by reference in their entirety.

“Paraoxonase activity” as used herein includes reference to one or more paraoxonase activity assays such as arylesterase assays, paraoxonase assays, lipolactonase assays, and equivalents thereof known to those skilled in the art, and so forth.

### Oxidized Paraoxonase 1 as a Risk Marker for Cardiovascular Disease

Provided herein are methods and markers for characterizing a subject's risk for developing CVD, having CVD, or experiencing a complication of CVD or major adverse cardiac event. In this context, such methods and markers are useful for characterizing a subject's risk of having vulnerable plaque or experiencing a myocardial infarction.

In one embodiment, the method comprises determining levels of one or more oxidized PON1-related biomolecules in a biological sample obtained from the subject. In one embodiment, at least one of the oxidized PON1-related biomolecules is an oxidized form of PON1. In another embodiment, at least one of the oxidized PON1-related biomolecules is an oxidized PON1 peptide fragment. Such fragment is three (3) or more amino acids in length and, except for the oxidized amino acid residues contained therein, comprises an amino acid sequence identical to all or a portion of SEQ ID NO: 1.

| | |
|---|---|
| 1 | MAKLTALTLL GLGLALFDGQ KSSFQTRFNV HREVTPVELP NCNLVKGVDN GSEDLEILPN |
| 61 | GLAFISSGLK YPGIMSFDPD KSGKILLMDL NEEDPVVLEL GITGNTLDIS SFNPHGISTF |
| 121 | TDEDNTVYLL VVNHPDSSST VEVFKFQEEE KSLLHLKTIR HKLLPSVNDI VAVGPEHFYA |
| 181 | TNDHYFADPY LKSWEMHLGL AWSFVTYYSP NDVRVVAEGF DFANGINISP DGKYVYIAEL |
| 241 | LAHKIHVYEK HANWTLTPLK SLDFDTLVDN ISVDPVTGDL WVGCHPNGMR IFYYDPKNPP |
| 301 | GSEVLRIQDI LSEEPKVTVV YAENGTVLQG STVAAVYKGK LLIGTVFHKA LYCEL |

The oxidized PON1 peptide fragments are at least three amino acids in length and may comprise a modified PON1 protein sequence, i.e., the peptide may comprise a sequence that, except for the presence of an oxidized amino acid, particularly an oxidized tyrosine residue, is identical to a sequence in SEQ ID NO: 1. In other embodiments, the oxidized PON1 peptide fragment is 4, 5, 6, 7, 8, 9, 10, 11, 12, 13, 14, 15, 16, 17, 18, 19, 20, 25, 30, 35, 40, 45, 50, 75, 100, 125, 150, 175, 200, 225, 250, 300, 350, or 355 amino acids in length. In other embodiments, the oxidized PON1 peptide fragment is 11-20, 21-30, 31-40, 41-50, 51-60, 61-70, 71-80, 81-90, 91-100, 101-110, 111-120, 121-130, 131-140, 141-150, 151-160, 161-170, 171-180, 181-190, 191-200, 201-210, 211-220, 221-230, 231-242, 250-300, 300-350, or 350-355 amino acids in length.

In certain embodiments such oxidized PON1 peptide fragments comprise one or more oxidized amino acids that indicate that the PON1 protein from which the peptide has been derived was oxidized by a myeloperoxidase (“MPO”)-related system. PON1 oxidation may take place by exposure to MPO-generated reactive chlorinating species (like those formed by the MPO/H₂O₂/Cl⁻ system, or HOCl), or MPO-related reactive nitrogen species (like those formed by the MPO/H₂O₂/NO₂⁻ system, or ONOO⁻), or alternative MPO-related oxidation pathways (e.g., MPO-generated tyrosyl radical generating systems). Thus, examples of suitable peptides include, but are not limited to, oxidized PON1 peptide fragments that comprise chlorotyrosine, nitrotyrosine, dityrosine, monohydroxytryptophan, dihydroxytryptophan, methionine sulfoxide, oxohistidine, trihydroxyphenylalanine, dihydroxyphenylalanine, tyrosine peroxide, or other oxidized amino acids formed by exposure of PON1 to MPO-generated oxidants. In some embodiments, the oxidized PON1 peptide fragment comprises at least one of oxidized tyrosine residue 71, 128, 179, 185, 190, 207, 208, 234, 236, 248, 293, 294, 321, 337, 352, oxidized tryptophan residue 194, 202, 254, 281, oxidized methionine residue 75, 88, 196, and 289. In certain embodiments the oxidized PON1 peptide fragment comprises at least one of oxidized tyrosine residue 71 and oxidized tryptophan position 254. In some embodiments the oxidized tyrosine residue is nitrotyrosine, chlorotyrosine, or dityrosine. In some embodiments the oxidized tryptophan residue is monohydroxytryptophan or dihydroxytryptophan. In some embodiments the oxidized methionine residue is methionine sulfoxide.

Exemplary sequences of PON1 peptides with potential oxidation sites are listed in Table 1. It is to be understood that the potential oxidation sites in the peptides of Table 1 may be flanked by 2, 3, 4, 5, 6, 7, 8, 9, 10, 11, 12, 13, 14, 15, 16, 17, 18, 19, 20, 25, 50, 75, 100, 125, 150, or 175 amino acids of SEQ ID NO: 1. In some embodiments the potential oxidation sites in the peptides of Table 1 may be flanked by amino acids only on the carboxy end or only the amino end, or both ends. In certain embodiments the potential oxidation sites in the peptides of Table 1 may be flanked by a different number of amino acids on the carboxy end than the amino end and vice versa. In certain embodiments the potential oxidation sites in the peptides of Table 1 may be flanked only on one end.

TABLE 1

| | Sequences of PON1 peptides with | |
|---|---|---|
| Residue | potential oxidization sites | |
| Positions | Sequence | Potential modification |
| 71 | SSGLK [symbol] PGIMS | 3-nitrotyrosine, |
| | | 3-chlorityrosine |
| 75 | KYPGI [symbol] SFDPD | Met oxidation |
| 88 | GKILL [symbol] DLNEE | Met oxidation |
| 128 | EDNTV [symbol] LLVVN | 3-nitrotyrosine, |
| | | 3-chlorityrosine |
| 179 | GPEHF [symbol] ATNDH | 3-nitrotyrosine, |
| | | 3-chlorityrosine |
| 185 | ATNDH [symbol] FADPY | 3-nitrotyrosine, |
| | | 3-chlorityrosine |
| 190 | YFADP [symbol] LKSWE | 3-nitrotyrosine, |
| | | 3-chlorityrosine |
| 194 | PYLKS [symbol] EMHLG | Trp oxidation |
| 196 | LKSWE [symbol] HLGLA | Met oxidation |
| 202 | LLGLA [symbol] SFVTY | Trp oxidation |
| 207, 208 | WSFVT [symbol] [symbol] SPNDV | 3-nitrotyrosine, |
| | | 3-chlorityrosine |
| 234 | SPDGK [symbol] VYIAEL | 3-nitrotyrosine, |
| | | 3-chlorityrosine |
| 236 | DGKYV [symbol] IAELL | 3-nitrotyrosine, |
| | | 3-chlorityrosine |
| 248 | HKIHV [symbol] EKHAN | 3-nitrotyrosine, |
| | | 3-chlorityrosine |
| 254 | KHAN [symbol] TLTPL | Trp oxidation |
| 281 | VTGDL [symbol] VGCHP | Trp oxidation |
| 289 | CHPNG [symbol] RIFYY | Met oxidation |
| 293, 294 | GMRIF [symbol] [symbol] DPKNP | 3-nitrotyrosine, |
| | | 3-chlorityrosine |
| 321 | KVTVV [symbol] AENGT | 3-nitrotyrosine, |
| | | 3-chlorityrosine |
| 337 | TVAAV [symbol] KGKLL | 3-nitrotyrosine, |
| | | 3-chlorityrosine |
| 352 | FHKAL [symbol] CEL | 3-nitrotyrosine, |
| | | 3-chlorityrosine |

Levels of the one or more oxidized PON1-related biomolecules in the bodily sample of the test subject may then be compared to a control value that is derived from levels of the one or more PON1-related biomolecules in comparable bodily samples of control subjects. In an alternative embodiment, levels of the one or more oxidized PON1-related biomolecules in the bodily sample of the test subject may then be compared to an internal standard based on levels of total PON1 and/or total PON1 activity. Furthermore, levels of one or more oxidized PON1-related biomolecules in a biological sample obtained from the subject may be compared as a ratio of specific oxidation products to unoxidized precursor, for example, the ratio of levels of oxidized PON1 to total PON1 and/or the ratio of oxidized PON1 activity to total PON1 activity. Test subjects whose levels of the one or more PON1-related biomolecules are above the control value or in the higher range of control values are at greater risk of having or developing cardiovascular disease than test subjects whose levels of the one more PON1-related biomolecules are at or below the control value or in the lower range of control values. Moreover, the extent of the difference between the subject's oxidized PON1-related biomolecule levels and the control value is also useful for characterizing the extent of the risk and thereby, determining which subjects would most greatly benefit from certain therapies.

In certain embodiments, the subject's risk profile for CVD is determined by combining a first risk value, which is obtained by comparing levels of one or more PON1-related biomolecules in a bodily sample of the subject with levels of said one or more PON1-related biomolecules in a control population, with one or more additional risk values to provide a final risk value. Such additional risk values may be obtained by procedures including, but not limited to, determining the subject's blood pressure, assessing the subject's response to a stress test, determining levels of myeloperoxidase, C-reactive protein, low density lipoprotein, or cholesterol in a bodily sample from the subject, or assessing the subject's atherosclerotic plaque burden.

In one embodiment, the method is used to assess the test subject's risk of having cardiovascular disease. Medical procedures for determining whether a human subject has coronary artery disease or is at risk for experiencing a complication of coronary artery disease include, but are not limited to, coronary angiography, coronary intravascular ultrasound (IVUS), stress testing (with and without imaging), assessment of carotid intimal medial thickening, carotid ultrasound studies with or without implementation of techniques of virtual histology, coronary artery electron beam computer tomography (EBTC), cardiac computerized tomography (CT) scan, CT angiography, cardiac magnetic resonance imaging (MRI), and magnetic resonance angiography (MRA). Because cardiovascular disease, typically, is not limited to one region of a subject's vasculature, a subject who is diagnosed as having or being at risk of having coronary artery disease is also considered at risk of developing or having other forms of CVD such as cerebrovascular disease, aortic-iliac disease, and peripheral artery disease. Subjects who are at risk of having cardiovascular disease are at risk of having an abnormal stress test or abnormal cardiac catherization. Subjects who are at risk of having CVD are also at risk of exhibiting increased carotid intimal medial thickness and coronary calcification, characteristics that can be assessed using non-invasive imaging techniques. Subjects who are at risk of having CVD are also at risk of having an increased atherosclerotic plaque burden, a characteristic that can be examined using intravascular ultrasound.

In another embodiment, the present methods are used to assess the test subject's risk of developing cardiovascular disease in the future. In one embodiment, the test subject is an apparently healthy individual. In another embodiment, the subject is not otherwise at elevated risk of having cardiovascular disease. In another embodiment, the present methods are used to determine if a subject presenting with chest pain is at risk of experiencing a heart attack or other major adverse cardiac event, such as a heart attack, a myocardial infarction, reinfarction, the need for revascularization, or death. As used herein, the term “near term” means within one year. Thus, subjects who are at near term risk may be at risk of experiencing a major adverse cardiac event within the following day, 3 months, or 6 months after presenting with chest pain. In another embodiment, the present methods are used to determine if a subject, particularly a human subject, is at risk of experiencing a major adverse cardiac event, e.g., heart attack or other major adverse cardiac event, such as a myocardial infarction, reinfarction, the need for revascularization, or death within the ensuing one, three, or ten years.

The present invention also provides a method for monitoring over time the status of CVD in a subject who has been diagnosed as having CVD. In this context, the method is also useful for monitoring the risk for atherosclerotic progression or regression in a subject with CVD. In one embodiment, the method comprises determining the levels of one or more of the oxidized PON1-related biomolecules in a biological sample taken from the subject at an initial time and in a corresponding biological sample taken from the subject at a subsequent time. An increase in levels of the one or more oxidized PON1-related biomolecules in a biological sample taken at the subsequent time as compared to the initial time indicates that the subject's CVD has progressed or worsened. A decrease in levels of the one or more oxidized PON1-related molecules indicates that the CVD has improved or regressed. For those subjects who have already experienced an acute adverse cardiovascular event, such as a myocardial infarction or ischemic stroke, such method can also be used to assess the subject's risk of having a subsequent acute adverse cardiovascular event. An increase over time in levels of the one or more oxidized PON1-related biomolecules in the subject indicates that a subject's risk of experiencing a subsequent adverse cardiovascular event has increased. A decrease over time in levels of the one or more oxidized PON1-related biomolecules in the subject indicates that the subject's risk of experiencing a subsequent adverse cardiovascular event has decreased.

In another embodiment, the present invention provides a method for evaluating therapy in a subject suspected of having or diagnosed as having cardiovascular disease. The method comprises determining levels of one or more oxidized PON1-related biomolecules, including oxidized PON1, an oxidized peptide fragment of PON1, and combinations thereof, in a biological sample taken from the subject prior to therapy and determining levels of the one or more of the oxidized apoA1 related biomolecules in a corresponding biological sample taken from the subject during or following therapy. A decrease in levels of the one or more oxidized PON1-related biomolecules in the sample taken after or during therapy as compared to levels of the one or more oxidized apoA1-related biomolecules in the sample taken before therapy is indicative of a positive effect of the therapy on cardiovascular disease in the treated subject.

### Biological Samples

Suitable biological samples useful for predicting or monitoring cardiovascular disease in a subject or for assessing the effect of therapeutic agents on subjects with cardiovascular disease include but are not limited to whole blood samples, samples of blood fractions, including but not limited to serum and plasma. The sample may be fresh blood or stored blood (e.g., in a blood bank) or blood fractions. The sample may be a blood sample expressly obtained for the assays of this invention or a blood sample obtained for another purpose which can be sub-sampled for the assays of this invention.

In one embodiment, the biological sample is whole blood. Whole blood may be obtained from the subject using standard clinical procedures. In another embodiment, the biological sample is plasma. Plasma may be obtained from whole blood samples by centrifugation of anti-coagulated blood. Such process provides a buffy coat of white cell components and a supernatant of the plasma. In another embodiment, the biological sample is serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation of apolipoprotein B containing proteins with dextran sulfate or other methods. Any of a number of standard aqueous buffer solutions, employing one of a variety of buffers, such as phosphate, Tris, or the like, at physiological pH can be used.

### Subjects

The subject is any human or other animal to be tested for characterizing its risk of CVD. In certain embodiments, the subject does not otherwise have an elevated risk of an adverse cardiovascular event. Subjects having an elevated risk of an adverse cardiovascular event include those with a family history of cardiovascular disease, elevated lipids, smokers, and/or prior acute cardiovascular event. (See e.g., Harrison's Principles of Experimental Medicine, 15th Edition, McGraw-Hill, Inc., N.Y.).

In certain embodiments the subject is an apparently healthy nonsmoker. “Apparently healthy,” as used herein, means individuals who have not previously been diagnosed as having any signs or symptoms indicating the presence of atherosclerosis, such as angina pectoris, history of an acute adverse cardiovascular event such as a myocardial infarction or stroke, evidence of atherosclerosis by diagnostic imaging methods including, but not limited to coronary angiography. Apparently healthy individuals also do not otherwise exhibit symptoms of disease. In other words, such individuals, if examined by a medical professional, would be characterized as healthy and free of symptoms of disease. “Nonsmoker” means an individual who, at the time of the evaluation, is not a smoker and has not used a tobacco product for the preceding 1 year period. This includes individuals who have never smoked as well as individuals who in the past have smoked but has not smoked for the past year.

### Immunoassays for Determining Levels of Oxidized PON1 and Oxidized PON1 Peptide Fragments

Levels of the oxidized PON1 and oxidized PON1 peptide fragments in the biological sample can be determined using polyclonal or monoclonal antibodies that are immunoreactive with such oxidized biomolecule. For example, antibodies immunospecific for nitrotyrosine containing oxidized PON1 peptide fragments may be made and labeled using standard procedures and then employed in immunoassays to detect the presence of such nitrotyrosine containing PON1 peptide in the sample. Suitable immunoassays include, by way of example, radioimmunoassays, both solid and liquid phase, fluorescence-linked assays, competitive immunoassays, and enzyme-linked immunosorbent assays. In certain embodiments, the immunoassays are also used to quantify the amount of the oxidized biomolecule that is present in the sample.

Monoclonal antibodies raised against the select oxidized polypeptide species are produced according to established procedures. Generally, the oxidized PON1 protein or PON1 peptide fragment is used to immunize a host animal.

Suitable host animals, include, but are not limited to, rabbits, mice, rats, goats, and guinea pigs. Various adjuvants may be used to increase the immunological response in the host animal. The adjuvant used depends, at least in part, on the host species. Such animals produce heterogeneous populations of antibody molecules, which are referred to as polyclonal antibodies and which may be derived from the sera of the immunized animals.

Monoclonal antibodies, which are homogenous populations of an antibody that bind to a particular antigen, are obtained from continuous cells lines. Conventional techniques for producing monoclonal antibodies are the hybridoma technique of Köhler, G. & Milstein, C., Nature 1975 Aug. 7; 256(5517):495-7 and the human B-cell hybridoma technique of Kozbor, D. & Roder, J. C., Immunology Today 1983 March; 4(3):72-9. Such antibodies may be of any immunoglobulin class including IgG, IgM, IgE, IgA, IgD, and any class thereof. Procedures for preparing antibodies against modified amino acids, such as, for example, 3-nitrotyrosine, are described in Ye, Y. Z., et al., Methods Enzymol. 1996; 269:201-9.

### Preparation of Antibodies

The oxidized PON1 protein or oxidized PON1 peptide fragment can be used as an immunogen to produce antibodies immunospecific for the oxidized protein or peptide fragment. The term “immunospecific” means the antibodies have substantially greater affinity for the oxidized PON1 protein or oxidized PON1 peptide fragment than for other proteins or polypeptides, including the unoxidized PON1 protein or unoxidized PON1 peptide fragment. Such antibodies may include, but are not limited to, polyclonal, monoclonal, chimeric, single chain, and FAb fragments.

The oxidized PON1 peptide fragments are at least three amino acids in length and comprise a modified PON1 protein sequence, i.e., the peptide comprises a sequence that, except for the presence of an oxidized amino acid, particularly an oxidized tyrosine residue, is identical to a sequence in SEQ ID NO: 1. In other embodiments, the oxidized PON1 peptide fragment is 4, 5, 6, 7, 8, 9, 10, 11, 12, 13, 14, 15, 16, 17, 18, 19, 20, 25, 30, 35, 40, 45, 50, 75, 100, 125, 150, 175, 200, 225, 250, 300, 350, or 355 amino acids in length. In other embodiments, the oxidized PON1 peptide fragment is 11-20, 21-30, 31-40, 41-50, 51-60, 61-70, 71-80, 81-90, 91-100, 101-110, 111-120, 121-130, 131-140, 141-150, 151-160, 161-170, 171-180, 181-190, 191-200, 201-210, 211-220, 221-230, 231-242, 250-300, 300-350, or 350-355 amino acids in length.

Peptides that are less than 6 amino acids in length conventionally are fused with those of another protein such as keyhole limpet hemocyanin and antibody chimeric molecule. Larger fragments, e.g., oxidized PON1 peptide fragments that are from 6 to 355 amino acids in length may also be used as the immunogen. The structure of larger immunogenic fragments of the oxidized PON1 protein can be determined using software programs, for example the MacVector program, to determine hydrophilicity and hydrophobicity, and ascertain regions of the protein that are likely to be present at the surface of the molecule.

Polyclonal antibodies are generated using conventional techniques by administering the oxidized PON1 protein or oxidized PON1 peptide fragment to a host animal. Depending on the host species, various adjuvants may be used to increase immunological response. Among adjuvants used in humans, Bacilli-Calmette-Guerin (BCG) and Corynebacterium parvum are preferable. Conventional protocols are also used to collect blood from the immunized animals and to isolate the serum and/or the IgG fraction from the blood.

For preparation of monoclonal antibodies, conventional hybridoma techniques are used. Such antibodies are produced by continuous cell lines in culture. Suitable techniques for preparing monoclonal antibodies include, but are not limited to, the hybridoma technique, the human B-cell hybridoma technique, and the EBV hybridoma technique.

Various immunoassays may be used for screening to identify antibodies having the desired specificity. These include protocols that involve competitive binding or immunoradiometric assays, and typically involve the measurement of complex formation between the respective oxidized PON1 polypeptide and the antibody.

The present antibodies may be used to detect the presence of or measure the amount of oxidized PON1 and oxidized PON1 peptide fragments in a biological sample from the subject. The method comprises contacting a sample taken from the individual with one or more of the present antibodies; and assaying for the formation of a complex between the antibody and a protein or peptide in the sample. For ease of detection, the antibody can be attached to a substrate such as a column, plastic dish, matrix, or membrane, preferably nitrocellulose. The sample may be a tissue or a biological fluid, including urine, whole blood, or exudate, preferably serum. The sample may be untreated, subjected to precipitation, fractionation, separation, or purification before combining with the antibody. Interactions between antibodies in the sample and the isolated protein or peptide are detected by radiometric, colorimetric, or fluorometric means, size-separation, or precipitation. Preferably, detection of the antibody-protein or peptide complex is by addition of a secondary antibody that is coupled to a detectable tag, such as for example, an enzyme, fluorophore, or chromophore. Formation of the complex is indicative of the presence of oxidized PON1 or oxidized PON1 peptide fragments in the individual's biological sample.

In certain embodiments, the method employs an enzyme-linked immunosorbent assay (ELISA) or a Western immunoblot procedure.

### Additional Methods for Measuring of Oxidized PON1 and Oxidized PON1 Peptide Fragments

Mass spectrometry-based methods (e.g., LC/ESI/MS/MS) may also be used to assess levels of oxidized PON1 and oxidized PON1 peptide fragments in the biological sample as shown in the examples below. Such methods are standard in the art and include, for example, HPLC with on-line electrospray ionization tandem mass spectrometry. Synthetic standard tryptic digests peptides for parent (unmodified) and modified (nitrated, chlorinated) forms can be made readily with automated peptide synthesizers using commercially available Fmoc modified amino acids. The parent molecules i.e., the PON1 and PON1 peptide fragments will have different masses than the oxidized molecules because of added moieties, added NO₂ or Cl⁻ moiety, for example). Thus, distinct parent-to-daughter ion transitions for each peptide would be achievable. Adding the nitro group to tyrosine changes the pKₐ of the phenoxy hydrogen on the tyrosine from 10 to 7. Thus, charge differences and changes in polarity between a modified and non-modified peptide have a high likelihood of showing distinct retention times on HPLC as well.

### Control Value

Levels of the oxidized PON1 and/or oxidized PON1 peptide fragment in the biological sample obtained from the test subject may be compared to a control value obtained from a reference cohort. In certain embodiments, the reference cohort is the general population. In certain embodiments, the reference cohort is a select population of human subjects. In certain embodiments, the reference cohort is comprised of individuals who have not previously had any signs or symptoms indicating the presence of atherosclerosis, such as angina pectoris, history of an acute adverse cardiovascular event such as a myocardial infarction or stroke, evidence of atherosclerosis by diagnostic imaging methods including, but not limited to coronary angiography. In certain embodiments, the reference cohort is comprised of individuals, who if examined by a medical professional would be characterized as free of symptoms of disease. In another example, the reference cohort may be individuals who are nonsmokers. “Nonsmoker,” as used herein, means an individual who, at the time of the evaluation, is not a smoker and has not used a tobacco product for the preceding 1 year period. This includes individuals who have never smoked as well as individuals who in the past have smoked but has not smoked for the past year. A nonsmoker cohort may have a different normal level of oxidized PON1 than will a smoking population or the general population. Accordingly, the control values selected may take into account the category into which the test subject falls. Appropriate categories can be selected with no more than routine experimentation by those of ordinary skill in the art. As further information becomes available as a result of routine performance of the methods described herein, population average values for oxidized PON1 levels may be used. In other embodiments, “normal” oxidized PON1 may be obtained by determining the oxidized PON1 levels in samples obtained from subjects without CVD, subjects who do not develop CVD in prescribed period of time, from archived patient samples, and the like.

The control value is related to the value used to characterize the level of the oxidized polypeptide obtained from the test subject. Thus, if the level of the oxidized polypeptide is an absolute value such as the units of oxidized PON1 per milliliter of blood, the control value is also based upon the units of oxidized PON1 per milliliter of blood in individuals in the general population or a select population of human subjects. Similarly, if the level of the oxidized PON1 or PON1 peptide fragment is a representative value such as an arbitrary unit obtained from a cytogram, the control value is also based on the representative value.

The control value can take a variety of forms. The control value can be a single cut-off value, such as a median or mean. The control value can be established based upon comparative groups such as where the risk in one defined group is double the risk in another defined group. The control values can be divided equally (or unequally) into groups, such as a low risk group, a medium risk group, and a high-risk group, or into quadrants, the lowest quadrant being individuals with the lowest risk the highest quadrant being individuals with the highest risk, and the test subject's risk of having CVD can be based upon which group his or her test value falls.

Control values of oxidized PON1 and/or oxidized PON1 peptide fragment in biological samples obtained, such as for example, mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G. & Miller, M. C., Clinical epidemiology and biostatistics, Malvern, Pa.: Williams & Wilkins; Harwal Pub. Co.; 1992 (ISBN 0683062069), which is specifically incorporated herein by reference. A “cutoff” value can be determined for each risk marker that is assayed.

### Comparison of Oxidized Biomolecule from the Test Subject to the Control Value

Levels of each select oxidized biomolecule, i.e., oxidized PON1 and/or oxidized PON1 peptide fragment, in the individual's biological sample may be compared to a single control value or to a range of control values. If the level of the present risk marker in the test subject's biological sample is greater than the control value or exceeds or is in the upper range of control values, the test subject is at greater risk of developing or having CVD than individuals with levels comparable to or below the control value or in the lower range of control values. In contrast, if levels of the present risk marker in the test subject's biological sample is below the control value or is in the lower range of control values, the test subject is at a lower risk of developing or having CVD than individuals whose levels are comparable to or above the control value or exceeding or in the upper range of control values. The extent of the difference between the test subject's risk marker levels and control value is also useful for characterizing the extent of the risk and thereby, determining which individuals would most greatly benefit from certain aggressive therapies. In those cases, where the control value ranges are divided into a plurality of groups, such as the control value ranges for individuals at high risk, average risk, and low risk, the comparison involves determining into which group the test subject's level of the relevant risk marker falls.

Alternatively, the level of oxidized biomolecule, i.e., oxidized PON1 or oxidized PON1 peptide fragment, may be compared to the level of an internal standard in the sample. Examples of such internal standards include, but are not limited to, levels of total PON1 and/or total PON1 activity. Furthermore, levels of one or more oxidized PON1-related biomolecules in a biological sample obtained from the subject may be compared as a ratio of specific oxidation products to unoxidized precursor, for example, the ratio of levels of oxidized PON1 to total PON1 and/or the ratio of oxidized PON1 activity to total PON1 activity.

The present predictive tests are useful for determining if and when therapeutic agents that are targeted at preventing CVD or for slowing the progression of CVD should and should not be prescribed for an individual. For example, individuals with values of oxidized PON1 above a certain cutoff value, or that are in the higher tertile or quartile of a “normal range,” could be identified as those in need of more aggressive intervention with lipid lowering agents and/or life style changes.

### Evaluation of CVD Therapeutic Agents

Also provided are methods for evaluating the effect of CVD therapeutic agents on individuals who have been diagnosed as having or as being at risk of developing CVD. Such therapeutic agents include, but are not limited to, anti-inflammatory agents, insulin sensitizing agents, antihypertensive agents, anti-thrombotic agents, anti-platelet agents, fibrinolytic agents, lipid reducing agents, direct thrombin inhibitors, ACAT inhibitor, CDTP inhibitor thioglytizone, glycoprotein IIb/IIIa receptor inhibitors, agents directed at raising or altering HDL metabolism such as PON1 milano or CETP inhibitors (e.g., torcetrapib), or agents designed to act as artificial HDL. Such evaluation comprises determining the levels of one or more oxidized PON 1-related biomolecules in a biological sample taken from the subject prior to administration of the therapeutic agent and a corresponding biological fluid taken from the subject following administration of the therapeutic agent. A decrease in the level of the selected risk markers in the sample taken after administration of the therapeutic as compared to the level of the selected risk markers in the sample taken before administration of the therapeutic agent is indicative of a positive effect of the therapeutic agent on cardiovascular disease in the treated subject.

### Kits

Also provided herein are kits for practicing the present methods. Such kits contain reagents for assessing levels of oxidized PON1, oxidized PON1 peptide fragments, or combinations thereof in a biological sample. In one embodiment, the reagent is an antibody that is immunospecific for oxidized PON1, or an oxidized PON1 peptide fragment, or both. In one embodiment, the kit also comprises instructions for using the reagent in the present methods. In another embodiment, the kit comprises information useful for determining a subject's risk of cardiovascular disease or a complication. Examples of such information include, but are not limited to, cut-off values, sensitivities at particular cut-off values, as well as other printed material for characterizing risk based upon the outcome of the assay. In some embodiments, such kits may also comprise control reagents, e.g., oxidized PON1, and/or oxidized PON1 peptide fragments.

### Paraoxonase 1/HDL Particle Number Ratio as a Risk Marker for Cardiovascular Disease

In certain embodiments, the PON1/HDL particle number ratio in a biological sample from a subject are compared to a control value that is derived from the PON1/HDL particle number ratio in comparable biological samples obtained from a control population. In certain embodiments, the present risk marker is a ratio of PON1 activity/HDL particle number (as determined by NMR). In some embodiments a surrogate for HDL particle number may be used. For example, levels of apolipoprotein A-1 (apoA1), apolipoprotein A-2 (apoA2), or (apoA1+apoA2) can serve as surrogates for HDL particle number. It should be understood that “HDL particle number” as used herein can be determined using levels of apoA1, apoA2, and (apoA1+apoA2). In another embodiment, the present methods comprise determining the ratio of PON1 mass to HDL particle number, apoA1, apoA2, or (apoA1+apoA2) in a biological sample, for example, blood, serum, or plasma, from the subject. In certain embodiments, the biological sample is blood, or a fluid derived from blood, e.g., serum, plasma, and/or urine. Levels of apoA1 and apoA2 can be measured using methods known to those skilled in the art, and include, but are not limited to, automated immunoanalysis and ELISA.

In one embodiment, the comparison characterizes the subject's present risk of having CVD, as determined using standard protocols for diagnosing CVD. Moreover, the extent of the difference between the subject's systemic PON1/HDL particle number ratio and the control value is also useful for characterizing the extent of the risk and thereby, determining which subjects would most greatly benefit from certain therapies. In another embodiment, the comparison characterizes the subject's risk of developing CVD in the future.

In another embodiment, the comparison can be used to characterize the subject's risk of experiencing a major adverse cardiac event, such as a myocardial infarction, the need for revascularization, stroke, congestive heart failure and/or death, within the ensuing three years. The present methods can also be used to determine if a subject presenting with chest pain is at risk of experiencing a major adverse cardiac event, such as a myocardial infarction, reinfarction, the need for revascularization, and/or death, near term, e.g., within the following day, 3 months, or 6 months after a subject presents with chest pain.

Also provided herein are methods for monitoring over time the status of CVD in a subject. In one embodiment, the method comprises determining the PON1/HDL particle number ratio in a biological sample taken from a subject at an initial time and in a corresponding biological sample taken from a subject at a subsequent time. An increase in the PON1/HDL particle number ratio in a biological sample taken at the subsequent time as compared to the initial time indicates that a subject's risk of having CVD has decreased. A decrease in the PON1/HDL particle number ratio indicates that the subject's risk of having CVD has increased. For those subjects who have already experienced a major adverse cardiovascular event, such as a myocardial infarction or ischemic stroke, such methods are also useful for assessing a subject's risk of experiencing a subsequent major adverse cardiovascular event. In such subjects, a decrease in levels of the PON1/HDL particle number ratio indicates that the subject is at increased risk of experiencing a subsequent major adverse cardiovascular event. An increase in the PON1/HDL particle number ratio in a subject over time indicates that the subject's risk of experiencing a subsequent major adverse cardiovascular event has decreased.

In another embodiment, the present invention provides a method for characterizing a subject's response to therapy directed at stabilizing or regressing CVD. The method comprises determining the PON1/HDL particle number ratio in a biological sample taken from the subject prior to therapy (or therapeutic lifestyle change such as diet or exercise), and determining the PON1/HDL particle number ratio in a corresponding biological sample taken from the subject during or following therapy or lifestyle change. An increase in the PON1/HDL particle number ratio in the sample taken after or during therapy or lifestyle change as compared to the PON1/HDL particle ratio in the sample taken before therapy is indicative of a positive effect of the therapy on cardiovascular disease in the treated subject.

In another embodiment, the present invention relates to kits that comprise reagents for assessing the PON1/HDL particle number ratio in biological samples obtained from a test subject. In certain embodiments, the kits also comprise printed materials such as instructions for practicing the present methods, or information useful for assessing a test subject's risk of CVD. Examples of such information include, but are not limited cut-off values, sensitivities at particular cut-off values, as well as other printed material for characterizing risk based upon the outcome of the assay. In some embodiments, such kits may also comprise control reagents.

In certain embodiments, the PON1/HDL particle ratio number in the bodily sample of the test subject is compared to a control value that is derived from the PON1/HDL particle number ratio in comparable bodily samples of control subjects. Test subjects whose PON1/HDL particle number ratio are below the control value or in the lower range of control values are at greater risk of having or developing cardiovascular disease than test subjects whose the PON1/HDL particle number ratio are at or above the control value or in the higher range of control values. Moreover, the extent of the difference between the subject's the PON1/HDL particle number ratio and the control value is also useful for characterizing the extent of the risk and thereby, determining which subjects would most greatly benefit from certain therapies.

In certain embodiments, the subject's risk profile for CVD is determined by combining a first risk value, which is obtained by comparing the PON1/HDL particle number ratio in a bodily sample of the subject with the PON1/HDL particle number ratio in a control population, with one or more additional risk values to provide a final risk value. Such additional risk values may be obtained by procedures including, but not limited to, determining the subject's blood pressure, assessing the subject's response to a stress test, determining levels of myeloperoxidase, homocitulline, C-reactive protein, low density lipoprotein, or cholesterol in a bodily sample from the subject, or assessing the subject's atherosclerotic plaque burden.

In one embodiment, the method is used to assess the test subject's risk of having cardiovascular disease. Medical procedures for determining whether a human subject has coronary artery disease or is at risk for experiencing a complication of coronary artery disease include, but are not limited to, coronary angiography, coronary intravascular ultrasound (IVUS), stress testing (with and without imaging), assessment of carotid intimal medial thickening, carotid ultrasound studies with or without implementation of techniques of virtual histology, coronary artery electron beam computer tomography (EBTC), cardiac computerized tomography (CT) scan, CT angiography, cardiac magnetic resonance imaging (MRI), and magnetic resonance angiography (MRA). Because cardiovascular disease, typically, is not limited to one region of a subject's vasculature, a subject who is diagnosed as having or being at risk of having coronary artery disease is also considered at risk of developing or having other forms of CVD such as cerebrovascular disease, aortic-iliac disease, and peripheral artery disease. Subjects who are at risk of having cardiovascular disease are at risk of having an abnormal stress test or abnormal cardiac catheterization. Subjects who are at risk of having CVD are also at risk of exhibiting increased carotid intimal medial thickness and coronary calcification, characteristics that can be assessed using non-invasive imaging techniques. Subjects who are at risk of having CVD are also at risk of having an increased atherosclerotic plaque burden, a characteristic that can be examined using intravascular ultrasound.

In another embodiment, the present methods are used to assess the test subject's risk of developing cardiovascular disease in the future. In one embodiment, the test subject is an apparently healthy individual. In another embodiment, the subject is not otherwise at elevated risk of having cardiovascular disease.

In another embodiment, the present methods are used to assess the test subject's risk of experiencing an adverse cardiac event within one, three, or ten years. In another embodiment, the present methods are used to determine if a subject presenting with chest pain is at risk of experiencing a heart attack or other major adverse cardiac event, such as a heart attack, a myocardial infarction, reinfarction, the need for revascularization, or death, near term after the subject presents with chest pain. As used herein, the term “near term” means within one year. Thus, subjects who are at near term risk may be at risk of experiencing a major adverse cardiac event within the following day, 3 months, or 6 months after presenting with chest pain.

The present invention also provides a method for monitoring over time the status of CVD in a subject who has been diagnosed as having CVD. In this context, the method is also useful for monitoring the risk for atherosclerotic progression or regression in a subject with CVD. In one embodiment, the method comprises determining the PON1/HDL particle number ratio in a biological sample taken from the subject at an initial time and in a corresponding biological sample taken from the subject at a subsequent time. A decrease in the PON1/HDL particle number ratio in a biological sample taken at the subsequent time as compared to the initial time indicates that the subject's risk for experiencing a major adverse event from the CVD has increased. An increase in the PON1/HDL particle ratio indicates that the subject's risk for experiencing a major adverse cardiac event from the CVD has improved or regressed. For those subjects who have already experienced an acute adverse cardiovascular event such as a myocardial infarction or ischemic stroke, such method can also be used to assess the subject's risk of having a subsequent major adverse cardiovascular event. A decrease over time in the PON1/HDL particle number ratio in the subject indicates that a subject's risk of experiencing a subsequent adverse cardiovascular event has increased. An increase over time in the PON1/HDL particle number ratio in the subject indicates that that the subject's risk of experiencing a subsequent adverse cardiovascular event has decreased.

In another embodiment, the present invention provides a method for evaluating therapy in a subject suspected of having or diagnosed as having cardiovascular disease. The method comprises determining the PON1/HDL particle number ratio in a biological sample taken from the subject prior to therapy and determining the PON1/HDL particle number ratio in a corresponding biological sample taken from the subject during or following therapy. An increase in the PON1/HDL particle number ratio in the sample taken after or during therapy as compared to the PON1/HDL particle number ratio in the sample taken before therapy is indicative of a positive effect of the therapy on cardiovascular disease in the treated subject.

### Biological Samples

Exemplary biological samples include, but are not necessarily limited to blood samples (e.g., blood, serum, plasma, and other blood-derived samples). The sample may be fresh blood or stored blood (e.g., in a blood bank) or blood fractions. The sample may be a blood sample expressly obtained for the assays of this invention or a blood sample obtained for another purpose which can be sub-sampled for the assays of this invention.

In one embodiment, the biological sample is whole blood. Whole blood may be obtained from the subject using standard clinical procedures. In another embodiment, the biological sample is plasma. Plasma may be obtained from whole blood samples by centrifugation of anti-coagulated blood such as heparin. However, in the case of PON1 activity measures the sample cannot include a metal chelator like EDTA since this inhibits PON1 activity measurements. Such process provides a buffy coat of white cell components and a supernatant of the plasma. In another embodiment, the biological sample is serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation of apolipoprotein B containing proteins with dextran sulfate or other methods. Any of a number of standard aqueous buffer solutions, employing one of a variety of buffers, such as phosphate, Tris, or the like, at physiological pH can be used.

### Subjects

The subject is any human or other animal to be tested for characterizing its risk of CVD. In certain embodiments, the subject does not otherwise have an elevated risk of an adverse cardiovascular event. Subjects having an elevated risk of an adverse cardiovascular event include those with a family history of cardiovascular disease, elevated lipids, smokers, prior acute cardiovascular event, etc. (See, e.g., Harrison's Principles of Experimental Medicine, 15th Edition, McGraw-Hill, Inc., N.Y.).

In certain embodiments the subject is apparently healthy. “Apparently healthy,” as used herein, means individuals who have not previously being diagnosed as having any signs or symptoms indicating the presence of atherosclerosis, such as angina pectoris, history of an acute adverse cardiovascular event such as a myocardial infarction or stroke, evidence of atherosclerosis by diagnostic imaging methods including, but not limited to coronary angiography. Apparently healthy individuals also do not otherwise exhibit symptoms of disease. In other words, such individuals, if examined by a medical professional, would be characterized as healthy and free of symptoms of disease.

In certain embodiments, the subject is a nonsmoker. “Nonsmoker” means an individual who, at the time of the evaluation, is not a smoker and has not had a tobacco product for the preceding 1 year period. This includes individuals who have never smoked as well as individuals who in the past have smoked but have not smoked for the past year. In certain embodiments, the subject is a smoker.

In some embodiments, the subject is a non-hyperlipidemic subject. A “non-hyperlipidemic” is a subject that is a non-hypercholesterolemic and/or a non-hypertriglyceridemic subject. A “non-hypercholesterolemic” subject is one that does not fit the current criteria established for a hypercholesterolemic subject. A non-hypertriglyceridemic subject is one that does not fit the current criteria established for a hypertriglyceridemic subject (See, e.g., Harrison's Principles of Experimental Medicine, 15th Edition, McGraw-Hill, Inc., N.Y.). Hypercholesterolemic subjects and hypertriglyceridemic subjects are associated with increased incidence of premature coronary heart disease. A hypercholesterolemic subject has an LDL level of >160 mg/dL, or >130 mg/dL and at least two risk factors selected from the group consisting of male gender, family history of premature coronary heart disease, cigarette smoking (more than 10 per day), hypertension, low HDL cholesterol (<40 mg/dL), diabetes mellitus, hyperinsulinemia, abdominal obesity, high lipoprotein (a), and personal history of cerebrovascular disease or occlusive peripheral vascular disease. A hypertriglyceridemic subject has a triglyceride (TG) level of >250 mg/dL. Thus, a non-hyperlipidemic subject is defined as one whose cholesterol and triglyceride levels are below the limits set as described above for both the hypercholesterolemic and hypertriglyceridemic subjects.

### Methods for Determining the PON1/HDL Particle Number Ratio

The level of PON1 in the subject's blood, serum, or plasma can be determined using any method for determining levels of enzymes in a subject's bodily fluid. In one embodiment, the level of PON1 refers to the activity level of PON1, as measured by established PON1 activity measures, such as paraoxonase, arylesterase, or various lipolactonase activity measures. In another embodiment, the level of PON1 refers to PON1 mass in the biological sample. PON1 mass levels in the biological sample can be determined using polyclonal or monoclonal antibodies that are immunoreactive with such protein. For example, antibodies immunospecific for PON1 may be made and labeled using standard procedures and then employed in immunoassays to determine apoA1 in the sample. Suitable immunoassays include, by way of example, radioimmunoassays, both solid and liquid phase, fluorescence-linked assays, competitive immunoassays, and enzyme-linked immunosorbent assays. In certain embodiments, the immunoassays are also used to quantify the amount of PON1 that is present in the sample.

Monoclonal antibodies raised against PON1 are produced according to established procedures. Generally, the PON1 protein is used to immunize a host animal. Suitable host animals, include, but are not limited to, rabbits, mice, rats, goats, and guinea pigs. Various adjuvants may be used to increase the immunological response in the host animal. The adjuvant used depends, at least in part, on the host species. Such animals produce heterogeneous populations of antibody molecules, which are referred to as polyclonal antibodies and which may be derived from the sera of the immunized animals.

Monoclonal antibodies, which are homogenous populations of an antibody that bind to a particular antigen, are obtained from continuous cells lines. Conventional techniques for producing monoclonal antibodies are the hybridoma technique of Köhler, G. & Milstein, C., Nature 1975 Aug. 7; 256(5517):495-7 and the human B-cell hybridoma technique of Kozbor, D. & Roder, J. C., Immunology Today 1983 March; 4(3):72-9. Such antibodies may be of any immunoglobulin class including IgG, IgM, IgE, IgA, IgD and any class thereof.

The term “high density lipoprotein” or “HDL” is defined in accordance with common usage of those of skill in the art. Generally “HDL” refers to a lipid-protein complex which when isolated by ultracentrifugation is found in the density range of d=1.063 to d=1.21. The concentration of HDL particles in a subject's blood, serum, or plasma can be determined by NMR. Alternatively, the concentration of HDL particles in a subject's blood, serum or plasma can be estimated by measuring the level of apoA1, apoA2, or (apoA1+apoA2) in the subject's blood, serum, or plasma. Apolipoprotein A-1 is the major structural protein on HDL. It consists of a series of amphipathic helices that are functionally important for protein-lipid interactions as well as protein-protein interactions. The carboxy terminus of ApoA1 has high lipid-binding capacity, while the amino terminus has limited lipid-binding capacity but may be important in protein-protein interaction (Frank, P. G. & Marcel, Y. L., J Lipid Res. 2000 June; 41(6):853-72). ApoA1 is largely responsible for mediating HDL assembly and is a determinant of HDL structure and composition. Levels of apoA1 and apoA2 in the subject's sample can be determined using polyclonal or monoclonal antibodies that are immunoreactive with such protein. For example, antibodies immunospecific for apoA1 may be made and labeled using standard procedures and then employed in immunoassays to determine apoA1 in the sample. Suitable immunoassays include, by way of example, radioimmunoassays, both solid and liquid phase, fluorescence-linked assays, competitive immunoassays, and enzyme-linked immunosorbent assays. In certain embodiments, the immunoassays are also used to quantify the amount of apoA1 that is present in the sample.

### Control Value

In certain embodiments, the PON1/HDL particle number ratio in the biological sample obtained from the test subject may be compared to a control value. The control value is based upon the PON1/HDL particle number ratio in comparable samples obtained from a reference cohort. In certain embodiments, the reference cohort is the general population. In certain embodiments, the reference cohort is a select population of human subjects. In certain embodiments, the reference cohort is comprised of individuals who have not previously had any signs or symptoms indicating the presence of atherosclerosis, such as angina pectoris, history of an acute adverse cardiovascular event such as a myocardial infarction or stroke, evidence of atherosclerosis by diagnostic imaging methods including, but not limited to coronary angiography. In certain embodiments, the reference cohort is comprised of individuals, who if examined by a medical professional would be characterized as free of symptoms of disease. In another example, the reference cohort may be individuals who are nonsmokers. “Nonsmoker”, as used herein, means an individual who, at the time of the evaluation, is not a smoker and has not used a tobacco product for the preceding 1 year period. This includes individuals who have never smoked as well as individuals who in the past have smoked but has not smoked for the past year. A nonsmoker cohort may have a different normal PON1/HDL particle number ratio than will a smoking population or the general population. Accordingly, the control values selected may take into account the category into which the test subject falls. Appropriate categories can be selected with no more than routine experimentation by those of ordinary skill in the art. As further information becomes available as a result of routine performance of the methods described herein, population average values for the PON1/HDL particle number ratio may be used. In other embodiments, “normal” PON1/HDL particle number ratios may be obtained by determining the PON1/HDL particle ratio in samples obtained from subjects without CVD, subjects who do not develop CVD in prescribed period of time, from archived patient samples, and the like.

The control value can take a variety of forms. The control value can be a single cut-off value, such as a median or mean. The control value can be established based upon comparative groups such as where the risk in one defined group is double the risk in another defined group. The control values can be divided equally (or unequally) into groups, such as a low risk group, a medium risk group and a high-risk group, or into quadrants, the lowest quadrant being individuals with the lowest risk the highest quadrant being individuals with the highest risk, and the test subject's risk of having CVD can be based upon which group his or her test value falls. Control values of the PON1/HDL particle number ratio in biological samples obtained, such as for example, mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G. & Miller, M. C., Clinical epidemiology and biostatistics, Malvern, Pa.: Williams & Wilkins; Harwal Pub. Co.; 1992 (ISBN 0683062069), which is specifically incorporated herein by reference. A “cutoff” value can be determined for each risk marker that is assayed.

### Comparison of the PON1/HDL Particle Number Ratio from the Test Subject to the Control Value

The PON1/HDL particle number ratio in the individual's biological sample may be compared to a single control value or to a range of control values. If the level of the present risk marker in the test subject's biological sample is greater than the control value or exceeds or is in the upper range of control values, the test subject is at lower risk of developing or having CVD than individuals with levels below the control value or in the lower range of control values. In contrast, if the PON1/HDL particle number ratio in the test subject's biological sample is below the control value or is in the lower range of control values, the test subject is at higher risk of developing or having CVD than individuals whose levels are comparable to or above the control value or in the upper range of control values. The extent of the difference between the test subject's risk marker levels and control value is also useful for characterizing the extent of the risk and thereby, determining which individuals would most greatly benefit from certain aggressive therapies. In those cases, where the control value ranges are divided into a plurality of groups, such as the control value ranges for individuals at high risk, average risk, and low risk, the comparison involves determining into which group the test subject's level of the relevant risk marker falls.

The present predictive tests are useful for determining if and when therapeutic agents that are targeted at preventing CVD or for slowing the progression of CVD should and should not be prescribed for an individual. For example, individuals with PON1/HDL particle number ratios below a certain cutoff value, or that are in the lower tertile or quartile of a “normal range,” could be identified as those in need of more aggressive intervention with lipid lowering agents, and/or life style changes.

### Evaluation of CVD Therapeutic Agents

Also provided are methods for evaluating the effect of CVD therapeutic agents on individuals who have been diagnosed as having or as being at risk of developing CVD. Such therapeutic agents include, but are not limited to, anti-inflammatory agents, insulin sensitizing agents, antihypertensive agents, anti-thrombotic agents, anti-platelet agents, fibrinolytic agents, lipid reducing agents, direct thrombin inhibitors, CDTP inhibitor thioglytizone, glycoprotein IIb/IIIa receptor inhibitors, agents directed at raising or altering HDL metabolism such as apoA1 milano or CETP inhibitors, or agents designed to act as artificial HDL. Such evaluation comprises determining the PON1/HDL particle number ratio in a biological sample taken from the subject prior to administration of the therapeutic agent and a corresponding biological fluid taken from the subject following administration of the therapeutic agent. An increase in the level of the selected risk markers in the sample taken after administration of the therapeutic as compared to the level of the selected risk markers in the sample taken before administration of the therapeutic agent is indicative of a positive effect of the therapeutic agent on cardiovascular disease in the treated subject.

### Kits

Also provided are kits for practicing the present methods. Such kits contain reagents for assessing levels of PON1 activity and/or mass and HDL particle number in a biological sample. In one embodiment, the kit comprises a reagent for measuring PON1 activity and a reagent, e.g., an antibody, for measuring apoA1 and/or apoA2 levels in the subject's bodily sample. In one embodiment, the kit also comprises instructions for using the reagent in the present methods. In another embodiment, the kit comprises information useful for determining a subject's risk of cardiovascular disease or a complication. Examples of such information include, but are not limited cut-off values, sensitivities at particular cut-off values, as well as other printed material for characterizing risk based upon the outcome of the assay.

### EXAMPLES

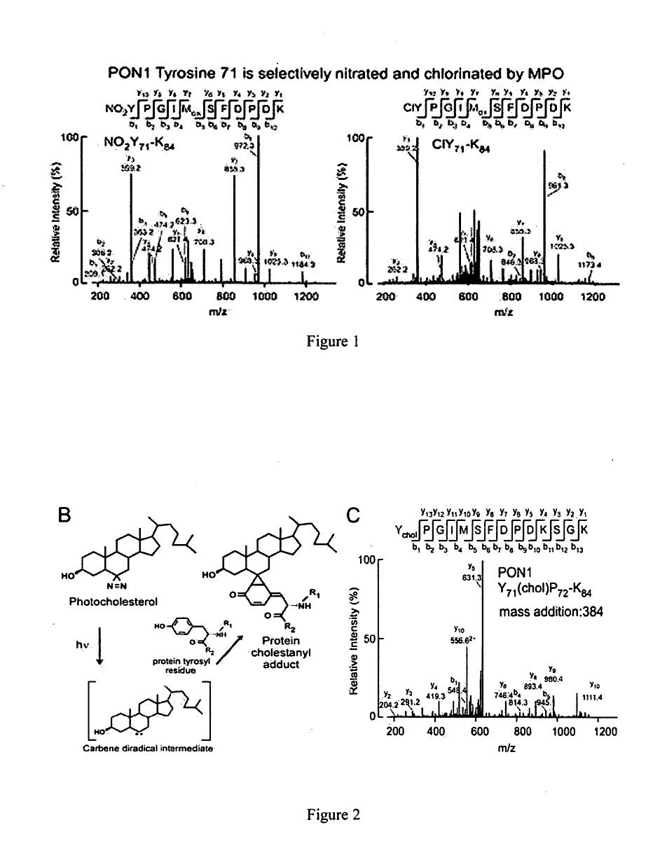

PON1 was isolated from human plasma (from subjects with known heart disease) using an antibody to PON1 and analyzed by LC/MS/MS. PON1 Tyr71 was identified as a residue that was unusually abundant as nitrotyrosine and chlorotyrosine in plasma of CAD subjects. Shown in FIG. 1 is a mass spectrum analysis of the tryptic peptide containing nitrated and chlorinated Tyr71 of PON1, the most abundant modification noted.

In order to define how structurally specific interactions between HDL and the associated proteins PON1 and MPO influence function of the interacting species, protein-protein interactions may be ascertained using cross-linking/proteomics studies, and HD-MS/MS studies, powerful tools for amino acid level of resolution determination of contact sites between proteins. However, few if any tools exist for determining where proteins like PON1 or MPO interact with the lipid components of HDL. These interactions may be critical for initial docking, or stabilizing of contacts. Indeed, lipid interactions are reportedly essential for maintaining PON1 catalytic activity. To address this need, we have developed novel photo-affinity tools, which when used in combination with proteomics studies, permit determination of precise interaction sites between the lipid phase of HDL and specific amino acid residues of HDL associated proteins. We have synthesized (confirmed by NMR) diazirine-labeled photoactivatable cholesterol analogues. Upon exposure to UV light, N₂ is eliminated generating a diradical carbene intermediate (Figure B). A reactive species scavenged at a diffusion-limited rate by interaction with H₂O. Thus, if the diradical carbene intermediate interacts with just one water molecule, it is quenched, and no adduct is formed. The diradical carbene also can interact with multiple protein targets, such as direct addition across double bonds, covalent addition to nucleophilic targets, and even adduction to amide bonds. Thus, mass spectrometry detection of a protein adduct indicates a site where protein-lipid interaction is tight enough to exclude water. Incubation of recombinant HDL (made with Photo-cholesterol) with PON1 led to discovery of a PON1 residue with a covalent cholestanyl adduct, Tyr71, the same residue identified as a preferred target for oxidation in vivo, and functional inactivation of PON1.

Novel diazirine photoaffinity label sterol and detection of a specific HDL lipid contact site on PON1. Structures of synthetic photoaffinity labeled cholesterol analog and scheme illustrating carbene diradical intermediate formed following UV light exposure, and presumed structure of tyrosine adduct (FIG. 2B). rHDL generated with Photo-cholesterol instead of cholesterol (1:100:10, apoA1, DMPC: Photo-cholesterol) was incubated with PON1 at 1:1 mol ratio and then briefly exposed to UV light. The mass spectrum shown is the tryptic peptide from PON1 identified with a photo-cholestanyl adduct. The target residue is identified as PON1 Tyr71 (FIG. 2C).

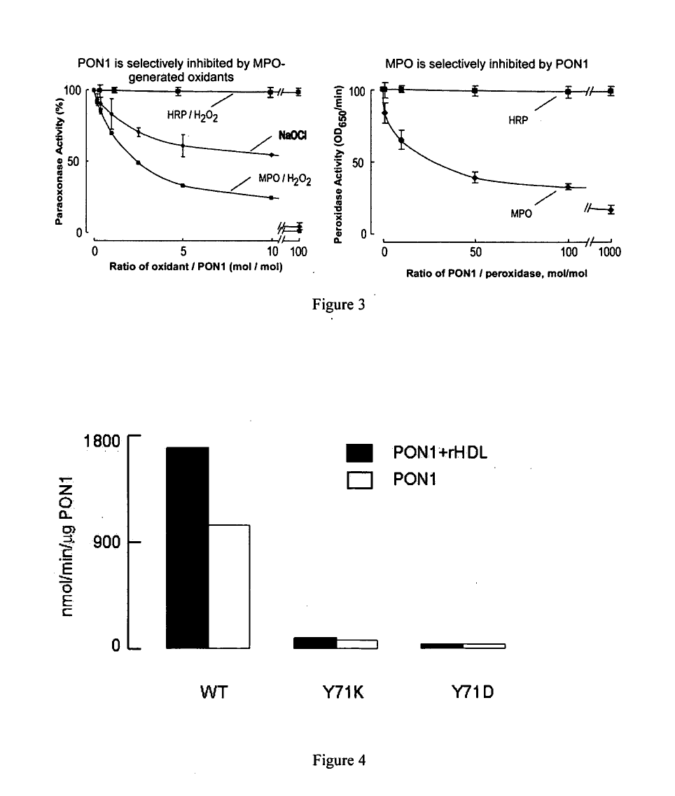

PON1 is selectively inhibited by MPO-generated oxidants under physiological conditions, and conversely, PON1 dose dependently and specifically modulates MPO activity. PON1 was dose dependently exposed to the MPO/H₂O₂/Cl⁻ system, hypochlorite, or the HRP/H₂O₂ system and paraoxonase activity determined (FIG. 3, left panel). Addition of isolated PON1 (RR192 isoform) to classic peroxidase activity assays of MPO and HRP reveal a striking preference for inhibition in MPO activity (FIG. 3, right panel).

MPO, HDL, and PON1 form a functional ternary complex that plays a role in the reciprocal regulation of MPO and PON1 activities. Using antibodies to MPO and stringent precipitation methods (high salt, detergents) we performed immunoaffinity proteomics studies of proteins that bind to MPO in plasma, and lesions. ApoA1 and PON1 were the two major proteins identified and were observed on gel by both Coomassie staining and Western blot. Subsequent proteomic, native gel, and gel filtration studies all support the existence of an isolatable ternary complex amongst MPO, PON1, and HDL in plasma. We therefore tested the hypothesis that exposure of PON1 to MPO-generated oxidants inhibit PON1 activity similar to the findings that MPO selectively targets apoA1 of HDL for oxidation and inactivation. The close proximity of MPO to PON1 on the HDL-MPO-PON1 ternary complex would limit the diffusion distance MPO generated oxidants must travel before hitting PON1, and therefore should make PON1 especially susceptible to oxidative inactivation in the presence of HDL. In the left panel of FIG. 3, low mole oxidant exposures to the MPO/H₂O₂/Cl⁻ system (and to a lesser extent reagent hypochlorite) result in PON1 activity being potently inhibited in the presence of HDL. In contrast, horseradish peroxidase (HRP), an alternative peroxidase that does not bind to HDL, failed to inhibit PON1 under comparable conditions (despite the presence of HDL). PON1 reciprocally inhibited MPO activity by approximately 50% (the PON1 RR192 isoform) but did not inhibit the peroxidase HRP (FIG. 3, right panel).

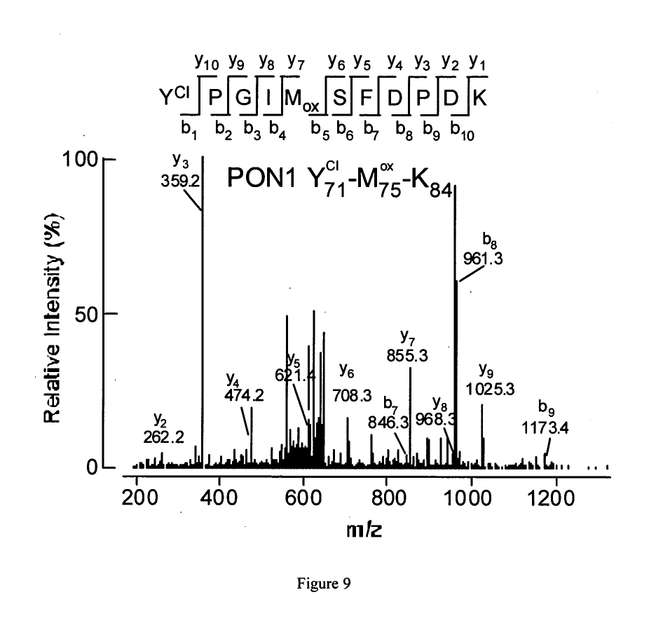

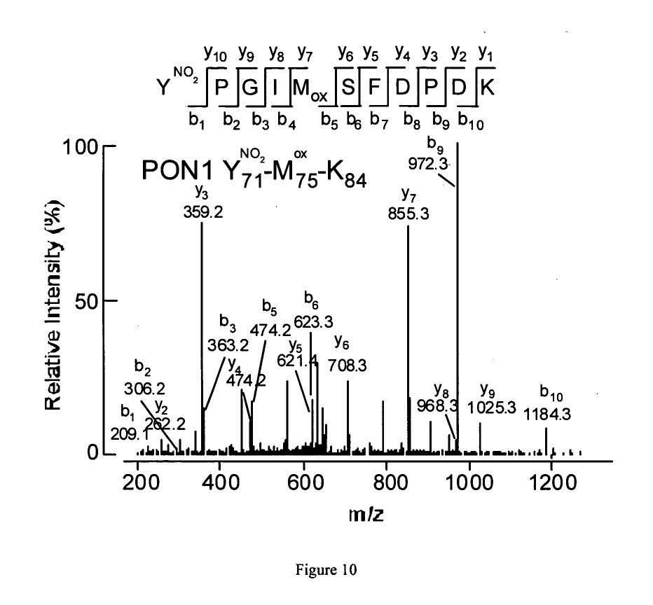

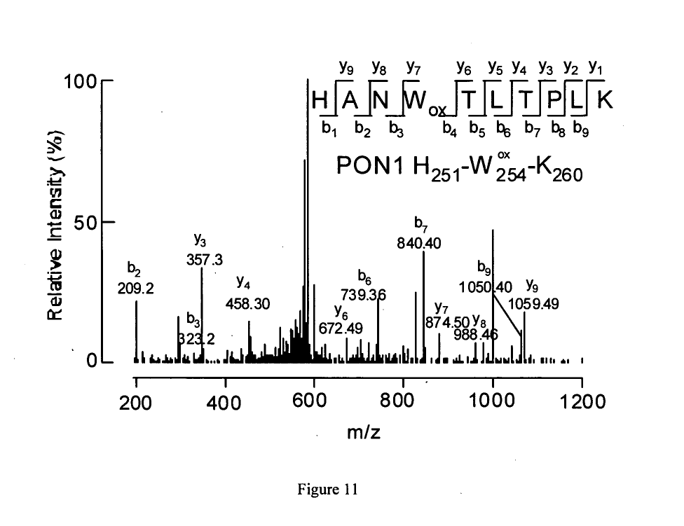

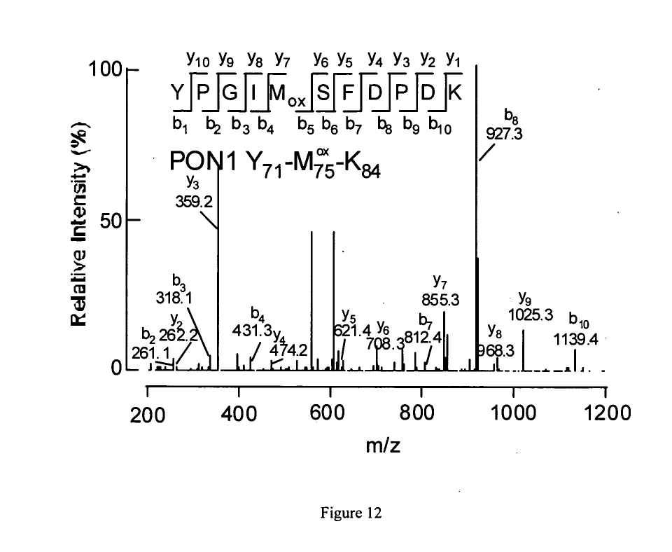

Tyrosine 71 of PON1 was identified by mass spectrometry studies as a selective target for nitration and chlorination in vivo (as monitored by LC/MS/MS based visualization of nitrotyrosine and chlorotyrosine in this specific site of PON1 recovered from atherosclerotic lesions) (FIG. 4). We therefore examined the functional significance of Tyr71 of PON1 by generating recombinant human PON1 as native (wild type) sequence, as well as with site directed mutagenesis of Tyr71 to either lysine (Y71K mutant) or aspartic acid (Y71D mutant). We then tested the impact of this mutation on PON1 activity in the presence and absence of HDL. PON1 is known to bind to HDL, and the complex demonstrates enhanced PON1 activity and stability. Moreover, in alternative studies we identified Tyr71 of PON1 as an important residue involved in PON1 interaction with HDL, and the stabilization/enhancement of PON1 activity when bound to HDL. Note that in WT PON1 there is PON1 activity (as measured using the paraoxonase activity assay) and the addition of HDL enhances this activity. Also note that mutation of the single amino acid (tyrosine 71) in PON1 to either lysine (K) or aspartic acid (D) ablates the activity of PON1. These results collectively show that PON1 amino acid Tyr71 is critical to PON1 activity. Since PON1 Tyr71 is also recognized as an endogenously modified residue in PON1 in human atherosclerotic lesions, these results suggest detection of oxidized Tyr71 of PON1 will serve as an excellent marker of functionally inactivated PON1 in vivo, and hence, increased cardiovascular risks. FIG. 6 shows tryptophan 254 of PON1 was identified by mass spectrometry studies as a selective target for oxidation in vivo (as monitored by LC/MS/MS based visualization of monohydroxytryptophan and dihydroxytryptophan in this specific site of PON1). FIG. 9 shows tyrosine 71 of PON1 was identified by mass spectrometry studies as a selective target for chlorination in vivo (as monitored by LC/MS/MS based visualization of chlorotyrosine in this specific site of PON1), and methionine 75 of PON1 was identified by mass spectrometry studies as a selective target for oxidation in vivo (as monitored by LC/MS/MS based visualization of methionine sulfoxide in this specific site of PON1). FIG. 10 shows tyrosine 71 of PON1 was identified by mass spectrometry studies as a selective target for nitration in vivo (as monitored by LC/MS/MS based visualization of nitrotyrosine in this specific site of PON1), and methionine 75 of PON1 was identified by mass spectrometry studies as a selective target for oxidation in vivo (as monitored by LC/MS/MS based visualization of methionine sulfoxide in this specific site of PON1). FIG. 11 shows tryptophan 254 of PON1 was identified by mass spectrometry studies as a selective target for oxidation in vivo (as monitored by LC/MS/MS based visualization of monohydroxytryptophan and dihydroxytryptophan in this specific site of PON1). FIG. 12 shows methionine 75 of PON1 was identified by mass spectrometry studies as a selective target for oxidation in vivo (as monitored by LC/MS/MS based visualization of methionine sulfoxide in this specific site of PON1).

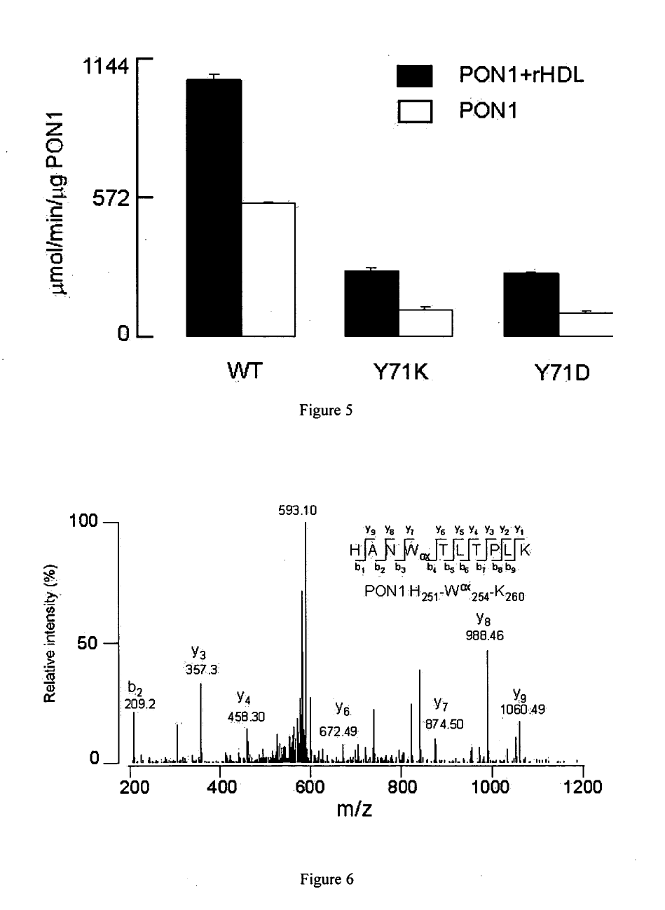

Studies in FIG. 5 are identical to those described in FIG. 4, examining the impact of site-directed mutagenesis of PON1 Tyr71 to either lysine or aspartic acid. Here, PON1 activity is monitored with arylesterase activity assay, an alternative more sensitive assay of PON1 activity. Note that both mutant forms of PON1 show impaired PON1 activity. Since Tyr71 is selectively oxidized in human lesions and PON1 activity is linked to CVD risks, these results again underscore the concept that detection of oxidized Tyr71 of PON1 in vivo will serve as a marker of cardiovascular risk.

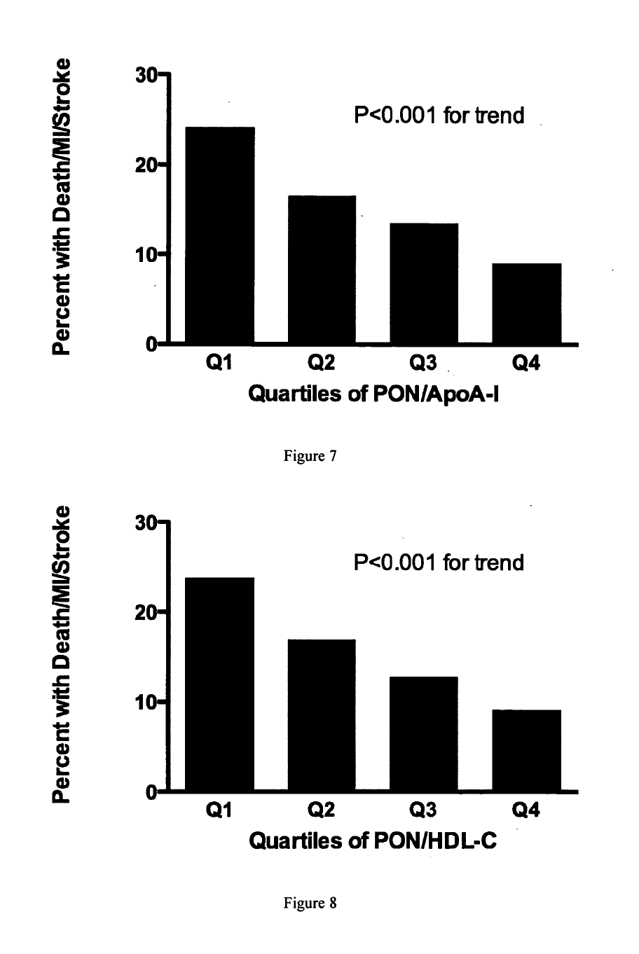

FIG. 7 shows plasma levels of PON1 activity and apoA1 were measured in 1399 sequential consenting patients who presented for a clinically indicated diagnostic coronary angiogram between September 2002 and November 2003 at the Cleveland Clinic. Patients were followed up until December 2006. The percentage of patients who experience a subsequent major adverse cardiovascular event (MACE: death, myocardial infarction or stroke) during the next 3 years stratified according to baseline quartiles of paraoxonase PON1/apoA1 is illustrated. This is the first demonstration that increasing levels of PON1/apoA1 was associated with a significant reduction in the likelihood of experiencing a clinical cardiovascular event. The ability of the amount of PON1 per HDL particle as demonstrated by PON1/apoA1 exceeds the predictive ability of PON1 activity alone and is demonstrated for PON1 activity measured by either paraoxonase or arylesterase activity. A similar finding is observed when apoA2 is measured in place of apoA1.

FIG. 8 shows plasma levels of PON1 activity and high-density lipoprotein cholesterol (HDL-C) were measured in 1399 sequential consenting patients who presented for a clinically indicated diagnostic coronary angiogram between September 2002 and November 2003 at the Cleveland Clinic. Patients were followed up until December 2006. The percentage of patients who experience a subsequent major adverse cardiovascular event (MACE: death, myocardial infarction or stroke) during the next 3 years stratified according to baseline quartiles of PON1/HDL-C is illustrated. This is the first demonstration that increasing levels of PON1/HDL-C was associated with a significant reduction in the likelihood of experiencing a clinical cardiovascular event. The ability of the amount of PON1 per HDL-C exceeds the predictive ability of PON1 activity alone and is demonstrated for PON1 activity measured by either paraoxonase or arylesterase activity.

What is claimed is:

1. A method for characterizing a subject's risk of having cardiovascular disease, comprising:
determining levels of one or more oxidized paraoxonase 1 (PON1)-related biomolecules in a biological sample from the subject, wherein the one or more oxidized PON1-related biomolecules are selected from oxidized PON1 and an oxidized PON1 peptide fragment,
comparing the levels of one or more oxidized paraoxonase 1 (PON1)-related biomolecules in a biological sample from the subject to a control value or an internal standard,
wherein the biological sample is blood, serum, or plasma,
wherein elevated levels of the one or more PON1-related oxidized biomolecules in the biological sample as compared to a control value or an internal standard indicates that the subject is at risk of having cardiovascular disease.

2. The method of claim 1, wherein the method employs a procedure or reagent for detecting oxidized PON1 or an oxidized PON1 peptide fragment that comprises one or more of the following amino acid residues: chlorotyrosine, nitrotyrosine, bromotyrosine, dityrosine, trihydroxyphenylalanine, dihydroxyphenylalanine, methionine sulfoxide, monohydroxytryptophan, dihydroxytryptophan, oxohistidine, and carbamyllysine.

3. The method of claim 2, wherein the one or more amino acid residues are an oxidative product of tyrosine residue 71, 128, 179, 185, 190, 207, 208, 234, 236, 248, 293, 294, 321, 337, 352, tryptophan residue 194, 202, 254, 281, methionine residue 75, 88, 196, and 289 of SEQ ID NO: 1.

4. The method of claim 1, wherein levels of the one or more oxidized PON1-related biomolecules are compared to a control value or a range of control values based upon levels of the one or more oxidized PON1-related biomolecules in comparable biological samples from a control population of human subjects.

5. A method for characterizing a subject's risk profile for cardiovascular disease, comprising:
determining a first risk value by comparing levels of one or more oxidized PON1-related biomolecules in a bodily sample from the subject to a control value; and
determining one or more additional cardiovascular risk values in the subject, wherein said one or more additional risk values are obtained by
a) determining the subject's blood pressure;
b) determining levels of low density lipoprotein, or cholesterol, or both in a biological sample from the subject;
c) assessing the subject's response to a stress test;
d) determining levels of myeloperoxidase, C-reactive protein, or both in a biological sample from the subject; and
e) determining the subject's atherosclerotic plaque burden, and
combining said first risk value with said one or more additional risk values to provide a final risk value.

6. A method for characterizing a subject's risk of developing cardiovascular disease or experiencing a major adverse cardiac event within 3 years, comprising:
determining levels of one or more oxidized PON1 related biomolecules in a biological sample from the subject, wherein the one or more PON1-related biomolecules are oxidized PON1 and an oxidized PON1 peptide fragment,
comparing levels of one or more oxidized PON1 related biomolecules in a biological sample from the subject to a control value or an internal standard,
wherein elevated levels of the one or more PON1-related oxidized biomolecules in the biological sample as compared to a control value or an internal standard indicates that the subject is at risk of developing cardiovascular disease or experiencing a major adverse cardiac event within 3 years.

7. The method of claim 6, wherein the method employs a procedure or reagent for detecting oxidized PON1 or an oxidized PON1 peptide fragment that comprises one or more of the following amino acid residues: chlorotyrosine, nitrotyrosine, bromotyrosine, dityrosine, trihydroxyphenylalanine, dihydroxyphenylalanine, methionine sulfoxide, monohydroxytryptophan, dihydroxytryptophan, oxohistidine, and carbamyllysine.

8. The method of claim 6, wherein the subject is an apparently healthy human subject.

9. The method of claim 6, wherein the subject is a non-smoker.

10. The method of claim 6, wherein the subject is not otherwise known to be at an elevated risk of having cardiovascular disease.

11. A method for characterizing the near term risk of experiencing a major adverse cardiac event in a subject who is presenting with chest pain, comprising:
determining levels of oxidized PON1 and/or an oxidized PON1 peptide fragment in a biological sample from the subject, and
comparing levels of oxidized PON1 and/or an oxidized PON1 peptide fragment in a biological sample from the subject to a control value or an internal standard,
wherein elevated levels of the one or more PON1-related oxidized biomolecules in the biological sample as compared to a control value or an internal standard indicates that the subject is at risk of experiencing a major cardiac event within the subsequent year.

12. The method of claim 11, wherein said biological sample is blood, serum, or plasma.

13. A method for evaluating therapy in a subject suspected of having or diagnosed as having cardiovascular disease, comprising:
determining levels of one or more oxidized PON1-related biomolecules in a biological sample taken from the subject prior to therapy and determining levels of the one or more of the oxidized PON1 related biomolecules in a corresponding biological sample taken from the subject during or following therapy,
comparing levels of one or more oxidized PON1-related biomolecules in a biological sample taken from the subject prior to therapy to levels of the one or more oxidized PON1-related biomolecules in the sample taken after or during therapy,
wherein a decrease in levels of the one or more oxidized PON1-related biomolecules in the sample taken after or during therapy as compared to levels of the one or more oxidized PON1-related biomolecules in the sample taken before therapy is indicative of a positive effect of the therapy on cardiovascular disease in the treated subject.

14. The method of claim 13, wherein said biological sample is blood, serum, or plasma.

15. A method of characterizing the risk of experiencing a subsequent acute cardiovascular event in a subject who has experienced one or more acute adverse cardiovascular events, comprising:
determining levels of one or more of the oxidized PON1-related biomolecules in a biological sample taken from the subject at an initial time and in a corresponding biological sample taken from the subject at a subsequent time,
comparing levels of one or more of the oxidized PON1-related biomolecules in a biological sample taken from the subject at an initial time to levels of the one or more oxidized PON1-related biomolecules in a biological sample taken at the subsequent time,
wherein an increase in levels of the one or more oxidized PON1-related biomolecules in a biological sample taken at the subsequent time as compared to the initial time indicates that a subject's risk of experiencing a subsequent adverse cardiovascular event has increased.

16. A method for monitoring over time the status of cardiovascular disease (CVD) in a subject with CVD, comprising:
determining the levels of one or more of the oxidized PON1-related biomolecules in a biological sample taken from the subject at an initial time and in a corresponding biological sample taken from the subject at a subsequent time,
comparing levels of one or more of the oxidized PON1-related biomolecules in a biological sample taken from the subject at an initial time to levels of the one or more oxidized PON1-related biomolecules in a biological sample taken at the subsequent time,
wherein an increase in levels of the one or more oxidized PON1-related biomolecules in a biological sample taken at the subsequent time as compared to the initial time indicates that a subject's CVD has worsened.

17. An antibody immunospecific for oxidized PON1, or an oxidized PON1 peptide fragment, wherein said oxidized PON1 peptide fragment is at least three amino acids in length and comprises one or more of the following amino acid residues: chlorotyrosine, nitrotyrosine, bromotyrosine, dityrosine, trihydroxyphenylalanine, dihydroxyphenylalanine, methionine sulfoxide, monohydroxytryptophan, dihydroxytryptophan, oxohistidine, and carbamyllysine.

18. The antibody of claim 17, wherein the oxidized PON1 protein or peptide fragment comprises at least one chlorotyrosine.

19. The antibody of claim 17, wherein the oxidized PON1 protein or peptide fragment comprises at least one nitrotyrosine.

20. The antibody of claim 17, wherein the oxidized PON1 protein or peptide fragment comprises at least one monohydroxytryptophan.

21. The antibody of claim 17, wherein the oxidized PON1 protein or peptide fragment comprises at least one dihydroxytryptophan.

22. The antibody of claim 17, wherein the oxidized PON1 protein or peptide fragment comprises at least one dityrosine.

23. The antibody of claim 17, wherein the oxidized PON1 protein or peptide fragment comprises at least one methionine sulfoxide.

24. The antibody of claim 17, wherein the oxidized PON1 protein or peptide fragment comprises at least one carbamyllysine or bromotyrosine.

25. A kit comprising:
one or more reagents for detecting oxidized PON1 or an oxidized PON1 peptide fragment,
at least one control selected from oxidized PON1 and an oxidized PON1 peptide fragment, and
one or more of the following printed materials: instructions for using said reagent in a method of assessing a test subject's risk of cardiovascular disease, information for assessing a test subjects risk cardiovascular disease, and recommendations for treating a subject who is determined to be at risk of cardiovascular disease.

26. The kit of claim 25, wherein at least one of the one or more reagents is an antibody that is immunospecific for oxidized PON1 or an oxidized peptide fragment of PON1, or both.

27. The kit of claim 25, wherein at least one of the one or more reagents is oxidized PON1 or an oxidized PON1 peptide fragment.

28. A method for characterizing a subject's risk of having cardiovascular disease, comprising:
determining the ratio of PON1 enzyme activity to HDL particle number in a biological sample from the subject,
comparing the ratio of PON1 enzyme activity to HDL particle number in a biological sample from the subject to a control value or an internal standard,
wherein the biological sample is blood, serum, or plasma
wherein a subject whose ratio of PON1 enzyme activity to HDL particle number is low compared to a control value or an internal standard indicates that the subject is at risk of having cardiovascular disease.

29. A method for characterizing a subject's risk of developing cardiovascular disease or experiencing a major adverse cardiac event within 3 years, comprising:
determining the ratio of PON1 activity to HDL particle number in a biological sample from the subject,
comparing the ratio of PON1 activity to HDL particle number in a biological sample from the subject to a control value or an internal standard,
wherein the biological sample is blood, serum, or plasma
wherein a subject whose ratio of PON1 activity per HDL particle number is low compared to a control value or an internal standard indicates that the subject is at risk of developing cardiovascular disease or experiencing a major adverse cardiac event within 3 years.

30. A method for characterizing the near term risk of experiencing a major adverse cardiac event in a subject who is presenting with chest pain, comprising:
determining the ratio of PON1 activity to HDL particle number in a biological sample from the subject,
comparing the ratio of PON1 activity to HDL particle number in a biological sample from the subject to a control value,
wherein the biological sample is blood, serum, or plasma
wherein a subject whose ratio of PON1 activity per HDL particle number is low compared to a control value indicates that the subject is at risk of experiencing a major cardiac event within the subsequent year.

31. A method for evaluating therapy in a subject suspected of having or diagnosed as having cardiovascular disease, comprising:
determining the ratio of PON1 activity to HDL particle number in a biological sample obtained from the subject prior to therapy and during or after therapy,
comparing the ratio of PON1 activity to HDL particle number in a biological sample obtained from the subject prior to therapy to the ratio of PON1 activity to HDL particle number in a biological sample obtained from the subject during or after therapy,
wherein the biological sample is blood, serum, or plasma,
wherein an increase in the PON1 activity to HDL particle number ratio in the subject's sample taken after or during therapy as compared to the PON1 activity to HDL particle number ratio in the sample taken before therapy is indicative of a positive effect of the therapy on cardiovascular disease in the treated subject.

32. A method of characterizing the risk of experiencing a subsequent acute cardiovascular event in a subject who has experienced one or more acute adverse cardiovascular events, comprising:
determining a PON1 activity per HDL particle number in a biological sample taken from the subject at an initial time and in a corresponding biological sample taken from the subject at a subsequent time,
comparing PON1 activity per HDL particle number in a biological sample taken from the subject at an initial time to PON1 activity per HDL particle number in a biological sample taken from the subject at a subsequent time,
wherein a decrease in the PON1 activity/HDL particle number ratio in a biological sample taken at the subsequent time as compared to the initial time indicates that a subject's risk of experiencing a subsequent adverse cardiovascular event has increased.

33. The method of claim 28, wherein the HDL particle number is determined using NMR.

34. The method of claim 28, wherein the HDL particle number is determined by determining apolipoprotein A-1 levels in the sample.

35. The method of claim 28, wherein the HDL particle number is determined by determining apolipoprotein A-2 levels in the sample.

36. The method of claim 28, wherein the HDL particle number is determined by determining apolipoprotein A-1 and apolipoprotein A-2 levels in the sample.

37. A method for characterizing a subject's risk of developing cardiovascular disease or experiencing a major adverse cardiac event within 1 year, comprising:
determining levels of one or more oxidized PON1 related biomolecules in a biological sample from the subject, wherein the one or more PON1-related biomolecules are oxidized PON1 and an oxidized PON1 peptide fragment,
comparing levels of one or more oxidized PON1 related biomolecules in a biological sample from the subject to a control value or an internal standard,
wherein elevated levels of the one or more PON1-related oxidized biomolecules in the biological sample as compared to a control value or an internal standard indicates that the subject is at risk of developing cardiovascular disease or experiencing a major adverse cardiac event within 1 year.

38. A method for characterizing a subject's risk of developing cardiovascular disease or experiencing a major adverse cardiac event within 10 years, comprising:
determining levels of one or more oxidized PON1 related biomolecules in a biological sample from the subject, wherein the one or more PON1-related biomolecules are oxidized PON1 and an oxidized PON1 peptide fragment,
comparing levels of one or more oxidized PON1 related biomolecules in a biological sample from the subject to a control value or an internal standard,
wherein elevated levels of the one or more PON1-related oxidized biomolecules in the biological sample as compared to a control value or an internal standard indicates that the subject is at risk of developing cardiovascular disease or experiencing a major adverse cardiac event within 10 years.

39. A method for characterizing a subject's risk of developing cardiovascular disease or experiencing a major adverse cardiac event within 1 year, comprising:
determining the ratio of PON1 activity to HDL particle number in a biological sample from the subject,
comparing the ratio of PON1 activity to HDL particle number in a biological sample from the subject to a control value or an internal standard,
wherein the biological sample is blood, serum, or plasma
wherein a subject whose ratio of PON1 activity per HDL particle number is low compared to a control value or an internal standard indicates that the subject is at risk of developing cardiovascular disease or experiencing a major adverse cardiac event within 1 year.

40. A method for characterizing a subject's risk of developing cardiovascular disease or experiencing a major adverse cardiac event within 10 years, comprising:
determining the ratio of PON1 activity to HDL particle number in a biological sample from the subject,
comparing the ratio of PON1 activity to HDL particle number in a biological sample from the subject to a control value or an internal standard,
wherein the biological sample is blood, serum, or plasma,
wherein a subject whose ratio of PON1 activity per HDL particle number is low compared to a control value or an internal standard indicates that the subject is at risk of developing cardiovascular disease or experiencing a major adverse cardiac event within 10 years.

41. The kit of claim 25, wherein at least one of the one or more reagents is an antibody that is immunospecific for at least one of chlorotyrosine, nitrotyrosine, bromotyrosine, dityrosine, trihydroxyphenylalanine, dihydroxyphenylalanine, methionine sulfoxide, monohydroxytryptophan, dihydroxytryptophan, oxohistidine, and carbamyllysine.
